SEQUENCE LISTING (12 sequences)

SEQ ID NO: 1 - protein, 186 aa, Homo sapiens [sequence omitted]

SEQ ID NO: 2 - protein, 163 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 3 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 4 - protein, 428 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 5 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 6 - protein, 134 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 7 - protein, 138 aa, Homo sapiens
IPFLEQNNFSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 8 - protein, 136 aa, Homo sapiens [sequence omitted]

SEQ ID NO: 9 - protein, 172 aa, Homo sapiens
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 10 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 11 - protein, 126 aa, Homo sapiens [sequence omitted]

SEQ ID NO: 12 - protein, 215 aa, Homo sapiens
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

(19) United States
(12) Patent Application Publication
(10) Pub. No.: US 2026/0328766 A1
(43) Pub. Date: Oct. 22, 2026

(54) POINT-OF-CARE ON-CHIP IGRA DEVICE AND METHOD FOR MEASURING SPECIFIC T CELL ACTIVATION

(71) Applicant: The Administrators of the Tulane Educational Fund, New Orleans, LA (US)

(72) Inventors: Ye Tony HU, New Orleans, LA (US); Bo NING, New Orleans, LA (US); Sutapa CHANDRA, New Orleans, LA (US)

(21) Appl. No.: 19/104,379

(22) PCT Filed: Aug. 18, 2023
(86) PCT No.: PCT/US2023/072511

§ 371 Date: Feb. 18, 2025

Related U.S. Application Data
(60) Provisional application No. 63/399,043, filed on Aug. 18, 2022.

(51) Int. Cl. G01N 33/569; G01N 33/53; G01N 33/68

(52) U.S. Cl. CPC G01N 33/5695; G01N 33/5304; G01N 33/6863; G01N 33/6869

(57) ABSTRACT

Method and device for detecting SARS-CoV-2 specific T-Cell interferon-gamma activation is described. By using a microfluidic chip to allow ELISpot interferon gamma release assays (IGRA) be performed using a small amount of fingerstick whole blood sample, fast results can be provided with minimal amount of blood sample.

### PRIOR RELATED APPLICATIONS

The application claims priority to U.S. App. No. 63/399,043, filed Aug. 18, 2022, which is incorporated by reference herein in its entirety for all purposes.

### FEDERALLY SPONSORED RESEARCH STATEMENT

Not applicable.

### FIELD OF THE DISCLOSURE

The disclosure generally relates to device and method for measuring specific T-cell activation, and more specifically to point-of-care on-chip IGRA device for measuring the T-cell activation by a pathogen, vaccine or therapy.

### BACKGROUND OF THE DISCLOSURE

IGRA assays measure either the amount of IFN-γ produced or the number of cells that secrete IFN-γ after stimulation with specific peptides derived from a pathogen of interest. The former approach relies on ELISA to detect IFN-γ secretion indicative of antigen-specific T cell induction, while the latter employs an enzyme-linked immunosorbent spot (ELISpot) assay to detect IFN-γ released and bound in proximity to activated T cells immobilized on a detection membrane. However, each of assays have drawbacks that limit their feasibility for routine use to estimate vaccine efficacy in large populations. Both ELISA-based and ELISpot-based IGRAs require significant amounts of fresh blood volumes, which should be processed for analysis within eight to fourteen hours after collection to obtain reliable results from viable target cells. ELISpot assays are more technically demanding than ELISA-based IGRAs since they require that peripheral blood mononuclear cells (PBMCs) be isolated from blood samples prior to stimulation with pathogen-specific target peptides and that the resulting PBMC culture reaction wells be scanned to quantify the number of colorimetric spots that indicate the number of responsive cells. However, both ELISpot and ELISA-based IGRAs can be technically demanding and are thus typically performed at central laboratories so that sample shipping logistics can be a limiting factor in assay performance.

Latent Mycobacterium tuberculosis (M tb) infections are estimated to affected one third of the world population, and have a 5-10% risk for progression to TB disease, which is responsible for 1.5 million deaths each year, more than any other infectious disease, except during the height of the COVID-19 pandemic. Individuals with latent M.tb-infections are at the greatest risk for TB disease within the first two years after M.tb infection but can remain at risk for disease progression throughout their lifetime. Addressing this latent disease reservoir is thus critical for the End TB Strategy to reduce TB incidence 90% by 2035 and eliminate TB by 2050.

Current methods used to diagnose latent M.tb infections include tuberculin skin tests (TSTs) and interferon gamma release assays (IGRAs) that can detect immune sensitization to M.tb-derived antigens, although each approach has shortcomings. Individuals screened using TST results must be evaluated 48-72 hours after a subcutaneous injection M.tb-derived material by a health care worker trained to evaluate such the resulting TST response. However, previous vaccination with M. bovis BCG, non-tuberculous mycobacteria infections that may have different treatment requirements, and incorrect TST interpretations can all produce false positive results that can lead to unnecessary or ineffective treatment. IGRAs do not require repeat visits and do not produce false positives for BCG-vaccinated individuals, but utilize whole blood samples that must be maintained under controlled conditions and used within ~16 hours of collection. The sensitivity of both tests is also reduced by factors that attenuate immune responses to their targeted M.tb antigens and are thus less reliable when used on individuals with compromised immune systems, recent M.tb infections, or recent live-virus measles or smallpox vaccinations, all of which can induce false negative results. HIV infection is a particular concern for M.tb screening efforts since HIV co-infected individuals are at increased risk for rapid progression from latent M.tb infection to TB, which is responsible for one third of HIV-related deaths. M.tb and HIV co-infection rates are also often high in regions with high endemic TB rates, and exceed 50% in parts of southern Africa.

CD4 T-cells play an essential role in the IFN-γ response induced following M.tb infection but IGRA results cannot reliably detect latent M.tb infections in HIV-infected individuals who have CD4 T-cells counts ≤200 cells/μL. We thus hypothesized that detection of activation markers increases that are less restricted to CD4 T-cells could enhance detection of M.tb-specific immune responses in HIV co-infected individuals, selecting OX40 (TNFRSF4/CD134) and 4-1BB (TNFRSF9/CD137) as candidate markers to detect M.tb infections in individuals with M.tb and HIV co-infections. OX40 is highly expressed activated versus nonactivated CD4 and CD8 T-cells and may thus serve as a more resilient marker of antigen-specific T-cell activation than CD4 T-cell IFN-γ expression. Similarly, 4-1BB expression is also induced on both activated CD4 and CD8 T-cells, although CD8 T cells can upregulate 4-1BB more rapidly and to a higher level than CD4 T cells. Further, since both these proteins are T-cell surface markers (TSMs) and thus can be directly detected with labeled specific antibodies, eliminating the need for the cell fixation and permeabilization steps required in intracellular cytokine staining assays.

Additionally, given the continuing emergence of SARS-CoV-2 variants of concern (VOCs), new approaches are needed to evaluate the degree and durability of immune protection, including protection against specific VOCs, after vaccination or infection. This information is of critical importance for rapid evaluation of vaccine effectiveness, vaccination guidelines, and public health decisions. It can also be used to identify vulnerable populations or individuals who require further precautions or interventions, including additional vaccine doses, and their response to these interventions. Several studies have shown that VOCs, including B.1.1.7 (Alpha), B1.351 (Beta), P.1 (Gamma), B.1.617.2 (Delta), and B.1.1.529 (Omicron), exhibit differential resistance to the immune responses produced by vaccinated and previously infected individuals. For example the spike protein of Omicron, currently the dominant VOC strain in the US, can evade neutralization by antibodies produced by vaccinated individuals and convalescent patients with 10- to 44-fold higher efficiency than the Delta spike protein, and its spike protein is resistant to neutralizing antibodies of convalescent patients and vaccinated individuals, which can be highly variable and rapidly decrease with time.

Robust data indicating how immune responses to SARS-CoV-2 VOCs change with time is essential to estimate their role in the durability of vaccine-mediated protection against these variants. However, the kinetics of antibody responses post-infection or -vaccination vary among different populations, and this data has shown limited clinical value when used to monitor vaccine efficacy over time. Evidence indicates that SARS-CoV-2-specific T-cell immune responses remain active after neutralizing antibody titers decrease. Studies with SARS-CoV-1 and MERS indicate INF-γ secretion in T-cells persists far longer than antibody responses or in the absence of antibody responses and SARS-CoV-2-specific T cell responses can be detected in the absence of detectable specific antibodies. For example, immunocompromised individuals can exhibit inadequate seroconversion rates and neutralizing antibody responses following SARS-CoV-2 vaccination, but still reveal significant virus-specific T-cell responses, including strong T cell immunity to Omicron. Notably, Omicron can evade specific neutralizing antibodies, but still activate T-cell responses induced by prior vaccination or infection, with one study indicating that 70-80% of the vaccine-induced CD4 and CD8 T-cell response to the spike protein of the reference strain was retained for Omicron. Several studies have therefore employed interferon-gamma (IFN-γ) release assays (IGRAs) to evaluate T-cell responses in vaccinated individuals and SARS-CoV-2 patients. Analysis of T-cell responses to emerging SARS-CoV-2 VOCs may allow rapid prospective evaluation of vaccine efficacy and inform the need for additional vaccine doses or the development of variant-specific vaccines.

New IGRA approaches that streamline the workflows, reduce the technical demands, and decrease the performance times of these assays are thus required to improve their utility for high-throughput analyses. Microfluidic techniques can simplify assay workflows, reduce sample volumes to decrease reagent costs, and minimize technical requirements and assay variation by automating key sample handling steps. Microfluidic approaches have recently been employed for COVID-19 diagnostic assays and several microfluidic sensing platforms for RT-PCR, antigen or antibody tests have received Emergency Use Authorization (EUA) approval from the FDA and been commercialized for COVID-19 diagnosis. We therefore hypothesized that a microfluidic IGRA could be developed to permit broad application of IGRAs to analyze T-cell responses that could be used to evaluate vaccine efficacy over time, including potential responses against emerging SARS-CoV-2 VOCs, as well as other pathogens and targets for immunotherapies.

### SUMMARY OF THE DISCLOSURE

Here we introduce the development and performance of a microfluidic ELISpot-based IGRA that provides point-of-care (POC) analysis of T-cell responses to various pathogens that are capable of eliciting T-cell response, for example, M. tb markers or SARS-CoV-2 target peptides. A method and microfluidic assay platform that requires less time, infrastructure and expertise than standard IGRAs is proposed herein. The method and assay platform for M. tb detection detects at least one of multiple M tb markers to improve its sensitivity in patient populations with impaired CD4 T-cell response and simplify its detection workflow. Similar method/assay platform is also proposed for detecting SARS-CoV-2 target peptides.

For M. tb, the results indicate that this approach can provide robust results within six hours, versus the 24-48 hours required by conventional IGRAs, on a TSM microchip device that employ fingerstick whole blood microsamples and integrates all assay steps on the assay chip without requiring laboratory equipment or special expertise.

For SARS-CoV-2, we found that microchip results were comparable to those produced by other immunoassay methods, including flow-cytometry and conventional ELISpot assay results, when evaluating the response of individuals who had or had not been vaccinated against or infected with SARS-CoV-2. This microfluidic chip assay provides results within 5 hours using fingerstick blood samples (~25 μL) thereby reducing both the sample-to-answer time and the sample handling and equipment requirements for IGRA. Notably, this assay is readable using a cellphone microscope permitting utilization in resource limited areas.

In one aspect of this disclosure, a method of identifying pathogen-specific T-cell activation using a microfluidic chip is described. The method comprises the steps of: a) obtaining a biological sample from a subject; b) introducing an incubation mixture into the biological sample, wherein the incubation mixture comprises (i) peptides of a pathogen of interest or peptides of a vaccine of interest; and (ii) an antibody binding specific to activated T-cells upon stimulation by the peptides of the pathogen of interest or by the peptides of the vaccine of interest; c) detecting presence of activated T-cells in the biological sample from step b); wherein the antibody in step b) is conjugated with an enzyme or a fluorescent molecule.

In another aspect of this disclosure, a point-of-care kit for identifying pathogen-specific T-cell response is described. The point-of-care kit comprises a microfluidic chip having a plurality of microfluidic channels connecting a sample inlet to a detection chamber, wherein the detection chamber is coated with poly-lysine or other cell attachment enhancing reagents, gelatin. The detection chamber can also be coated with T-cell specific antibodies such as anti-CD4 and anti-CD8 antibodies in order to better capture CD4+ and CD8+ T-cells within PBMCs. The microfluidic channels allow sufficient time to enhance the T-cell response against antigen peptides, while speeding up cytokine release of T-cell activation surface marker expression.

In another aspect of this disclosure, a method of identifying pathogen-specific T-cell activation using a microfluidic chip described herein is disclosed. The method comprises: a) obtaining a biological sample from a subject; b) introducing an incubation mixture into the biological sample, wherein the incubation mixture comprises (i) peptides of a pathogen of interest or peptides of a vaccine of interest, and (ii) an antibody specific to a cytokine or a surface marker, wherein the cytokine is secreted by T-cells in the biological sample upon stimulation by the peptides of the pathogen of interest or by the peptides of the vaccine of interest; c) introducing the biological sample from step b) into the detection chamber in the microfluidic chip through the sample inlet; and d) detecting presence of the cytokine or the surface marker in the detection chamber; wherein the cytokine- or surface marker-specific antibody is conjugated with an enzyme or a fluorescent molecule.

In one embodiment, the antibody binding specific to activated T-cells are anti-human interferon-gamma antibodies, or antibodies binding specific to cytokines or surface markers expressed by activated T-cells.

In one embodiment, the anti-human interferon-gamma antibodies target and bind to interferon gamma secreted by T-cells after being activated by peptides coming from a pathogen of interest. The anti-human interferon-gamma antibodies are conjugated with an enzyme or fluorescent molecules in order to generate visual signals upon binding to interferon gamma. The fluorescent signals to indicate the presence of IFN-γ.

In one embodiment, the method further comprising step b-1): obtaining T-cells in the whole blood sample by CD4 and CD8 specific antibodies.

In one embodiment, the point-of-care kit can further comprise an incubation container within which peptides from a pathogen of interest are present. The collected biological sample can be introduced into the incubation container and incubate with the peptides in order for the T-cells to be activated.

In one embodiment, the method comprises an activation step, wherein the T-cells in the biological sample is activated by the pathogen-specific peptides or vaccines of interest or markers for a predetermined period of time.

In one embodiment, the activation step lasts about 1 to 6 hours. In another embodiment, the activation step lasts about 2 to 6 hours.

In one embodiment, the incubation container further comprises phorbol 12-myristate13-acetate (PMA) and ionomycin for T-cell stimulation. In another embodiment, the fluorescent molecule is Fluorescein isothiocyanate (FITC) or Alexa Fluor® 488.

In one embodiment, the pathogen of interest is SARS-CoV-2, HIV or M. tuberculosis. Other pathogens may elicit T-cell responses can also be detected. Non-limiting pathogens include Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, and Trypanosoma cruzi.

In one embodiment, treatment response of interest is vaccines (SARS-CoV-2 vaccine) may elicit T cell response. Non-limiting vaccines include Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, and Trypanosoma cruzi.

Other antigen-specific T-cell response evaluation assay comprises CAR-T, TCR-T therapy, PD-1/PD-L1, CTLA-4, TIM3, LAG3 or other checkpoint blockage treatment in cancer. The method and device of this disclosure can include antibodies against these immune checkpoint proteins and screen for corresponding T-cell responses.

In one embodiment, the pathogen of interest is M. tb and the peptide or markers that can be used to capture the activated T-cells comprises at least a portion of OX-40, 4-1BB, CD59, LAG-3, TIM3, and _IL-12R, CD28, CD57, KIR, KLRG-1, CD27, PD-1, CTLA-4, IFN-γ, IL-2, IL-10, TNF-α. In one embodiment, the peptide or makers are extracellular domain of OX-40, 4-1BB, CD59, LAG-3, TIM3, and _IL-12R, CD28, CD57, KTR, KLRG-1, CD27, PD-1, CTLA-4, IFN-γ, IL-2, IL-10, TNF-α.

In one embodiment, the detection chamber in the microfluidic is treated with CD4- or CD8-specific antibodies. In one embodiment, the CD4- or CD8-specific antibodies are treated via EDC-NHS chemistry.

In one embodiment, the detection chamber is coated with poly-lysine, and the concentration of the poly-lysine can range from 1 μg/mL to 100 μg/mL. In another embodiment, the concentration of the poly-lysine can range from 5 μg/mL to 50 μg/mL.

In one embodiment, the microfluidic channel has a width between 10 μm and 200 μm, and a height between 10 μm and 200 μm. In another embodiment, the microfluidic channel has a width between 50 μm and 150 μm, and a height between 50 μm and 150 μm. In another embodiment, the microfluidic channel has a width of about 100 gpm, and a height of about 100 μm.

In one embodiment, the detection chamber has a size of 1 to 10 mm². In one embodiment, the detection chamber has a size of 10 mm×3 mm×0.1 mm.

In one embodiment, the biological sample is whole blood sample from fingerstick. This is different from the conventional ELISpot where peripheral blood mononuclear cells (PBMCs) must first be isolated before being subject to the test.

In one embodiment, the amount of the whole blood sample is less than 1 mL. In one embodiment, the amount of the whole blood sample is less than 100 μL. In another embodiment, the amount of the whole blood sample is less than 50 μL.

In one embodiment, the peptides of the pathogen of interest include SARS-CoV-2 spike peptide pool and BEI NR-52402. However, other peptide pools can also be used, as long as they represent the commonly encountered peptides from the pathogen that may elicit T-cell specific response. Non-limiting examples include peptides from Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, Trypanosoma cruzi, or tumor specific antigen peptide from NY-ESO-1, HER2, PSA, TRP-2, EpCAM, GPC3, mesothelin (MSLN), MUC1 and EGFR.

In one embodiment, the first interferon-gamma-specific antibody is M700-A from Endogen. However, other interferon-gamma-specific antibody can also be used.

In one embodiment, the mixture of the biological sample and the incubation mixture is introduced into the reaction chamber of the point-of-care device at a flow rate between 5 μl/min to 20 μl/min.

In one embodiment, the cytokine is IL-2, IL-4, IL-17 or TNFα. However, other cytokines may also be used.

In one embodiment, the surface marker is at least a portion of OX-40, 4-1BB, CD59, LAG-3, TIM3, and IL-12R, CD28, CD57, KIR, KLRG-1, CD27, PD-1, CTLA-4, IFN-γ, IL-2, IL-10, or TNF-α.

In one embodiment, the portion of OX-40 is the extracellular domain of OX-40, having the following amino acid sequence: LH CVGDTYPSND RCCHECRPGN GMVSRCSRSQ NTVCRPCGPG FYNDVVSSKP CKPCTWCNLR SGSERKQLCT ATQDTVCRCR AGTQPLDSYK PGVDCAPCPP GHFSPGDNQA CKPWTNCTLA GKIHTLQPASN SSDAICEDRD PPATQPQETQ GPPARPITVQ PTEAWPRTSQ GPSTRPVEVP GGRA (SEQ ID NO. 1).

In one embodiment, the portion of 4-1BB is the extracellular domain of 4-1BB, having the following amino acid sequence: LQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQ (SEQ ID NO. 2).

In one embodiment, the portion of CD59 is the extracellular domain of CD59, having the following amino acid sequence: MGIQGGSVLFGLLLVLAVFCHSGHSL QCYNCPNPTADCKTAVNCSSDFDACLITKAGLQVYNKCWKFEHCNFNDVTTRLRENEL TYYCCKKDLCNFNEQLENGGTSLSEKTVLLLVTPFLAAAWSLHP (SEQ ID NO. 3).

In one embodiment, the portion of LAG-3 is the extracellular domain of LAG-3, having the following amino acid sequence: LQPGAEVPVVWA QEGAPAQLPCSPTIPLQDLSLLRRAGVTWQHQPDSGPPAAAPGHPLAPGPHPAAPSSWG PRPRRYTVLSVGPGGLRSGRLPLQPRVQLDERGRQRGDFSLWLRPARRADAGEYRAAV HLRDRALSCRLRLRLGQASMTASPPGSLRASDWVILNCSFSRPDRPASVHWFRNRGQGR VPVRESPHHHLAESFLFLPQVSPMDSGPWGCILTYRDGFNVSIMYNLTVLGLEPPTPLTV YAGAGSRVGLPCRLPAGVGTRSFLTAKWTPPGGGPDLLVTGDNGDFTLRLEDVSQAQA GTYTCHIHLQEQQLNATVTLAIITVTPKSFGSPGSLGKLLCEVTPVSGQERFVWSSLDTPS QRSFSGPWLEAQEAQLLSQPWQCQLYQGERLLGAAVYFTELSSPGAQRSGRAPGALPA GHL (SEQ ID NO. 4)

In one embodiment, the portion of TIM3 is the extracellular domain of TIM3 having the following amino acid sequence: MTPWLGLIVLLGSWSLGDWGAEACTCSP SHPQDAFCNSDIVIRAKVVGKKLVKEGPFGTLVYTIKQMKMYRGFTKMPHVQYIHTEA SESLCGLKLEVNKYQYLLTGRVYDGKMYTGLCNFVERWDQLTLSQRKGLNYRYHLGC NCKIKSCYYLPCFVTSKNECLWTDMLSNFGYPGYQSKHYACIRQKGGYCSWYRGWAP PDKSIINATDP (SEQ ID NO. 5).

In one embodiment, the portion of CD28 is the extracellular domain of CD28, having the following amino acid sequence: NKILVKQSPMLVAYDNAVNLSCKYSYNLFSR EFRASLHKGLDSAVEVCVVYGNYSQQLQVYSKTGFNCDGKLGNESVTFYLQNLYVNQ TDIYFCKIEVMYPPPYLDNEKSNGTIIHVKGKHLCPSPLFPGPSKP (SEQ ID NO. 6).

In one embodiment, the portion of KIR is the extracellular domain of KIR, having the following amino acid sequence: IPFLEQNNFSPNTRTQKARHCGHCPEEWITYSNSCYYI GKERRTWEESLLACTSKNSSLLSIDNEEEMKFLASILPSSWIGVFRNSSHHPWVTINGLAF KHKIKDSDNAELNCAVLQVNRLKSAQCGSSMIYHCKHKL (SEQ ID NO. 7).

In one embodiment, the portion of KLRG-1 is the extracellular domain of KLRG-1, having the following amino acid sequence: LCQGSNYSTCASCPSCPDRWMKYGNHCY YFSVEEKDWNSSLEFCLARDSHLLVITDNQEMSLLQVFLSEAFCWIGLRNNSGWRWED GSPLNFSRISSNSFVQTCGAINKNGLQASSCEVPLHWVCKKCPFADQALF (SEQ ID NO. 8).

In one embodiment, the portion of CD27 is the extracellular domain of CD27, having the following amino acid sequence: ATPAPKSCPERHYWAQGKLCCQMCEP GTFLVKDCDQHRKAAQCDPCIPGVSFSPDHHTRPHCESCRHCNSGLLVRNCTITANAEC ACRNGWQCRDKECTECDPLPNPSLTARSSQALSPHPQPTHLPYVSEMLEARTAGHMQT LADFRQLPARTLSTHWPPQRSLCSSDFIR (SEQ ID NO. 9).

In one embodiment, the portion of PD-1 is the extracellular domain of PD-1, having the following amino acid sequence: FLDSPDRPWNPPTFSPALLVVTEGDNATFTCSFSNT SESFVLNWYRMSPSNQTDKLAAFPEDRSQPGQDCRFRVTQLPNGRDFHMSVVRARRND SGTYLCGAISLAPKAQIKESLRAELRVTERRAEVPTAHPSPSPRPAGQFQTLV (SEQ ID NO. 10).

In one embodiment, the portion of CTLA-4 is the extracellular domain of CTLA-4, having the following amino acid sequence: KAMHVAQPAVVLASSRGIASFVC EYASPGKATEVRVTVLRQADSQVTEVCAATYMMGNELTFLDDSICTGTSSGNQVNLTIQ GLRAMDTGLYICKVELMYPPPYYLGIGNGTQIYVIDPEPCPDSD (SEQ ID NO. 11).

In one embodiment, the portion of IL2Ra is the extracellular domain of IL2Ra, having the following amino acid sequence: ELCLYDPPEVPNATFKALSYKNGTILNCECK RGFRRLKELVYMRCLGNSWSSNCQCTSNSHDKSRKQVTAQLEHQKEQQTTTDMQKPT QSMHQENLTGHCREPPPWKHEDSKRIYHFVEGQSVHYECIPGYKALQRGPAISICKMKC GKTGWTQPQLTCVDEREHHRFLASEESQGSRNSSPESETSCPITTTDFPQPTETTAMTETF VLTMEYK (SEQ ID NO. 12).

In one embodiment, the anti-4-1BB antibody herein is Cd137 (4-1BB) Monoclonal Antibody (4B4 (4B4-1)), FITC, eBioscience 11-1379-42.

In one embodiment, the anti-OX-40 antibody used herein is Cd134 (OX40) Monoclonal Antibody (ACT35 (ACT-35)), FITC, eBioscience 11-1347-42.

As used herein, “cytokine” refers to any of a number of substances, such as interferon, interleukin, and growth factors, which are secreted by certain cells of the immune system and have an effect on other cells.

As used herein, “surface marker” refers to special proteins or peptides expressed on the surface of cells or carbohydrates attached to the cell membrane that often conveniently serve as markers of specific cell types.

As used herein, “interferon-gamma” or “IFN-7” refers to a dimerized soluble cytokine that belongs to the type II interferons. IFN-γ is critical for innate and adaptive immunity against viral, some bacterial and protozoan infections. IFN-γ is an important activator of macrophages and inducer of major histocompatibility complex class II molecule expression. IFN-γ is produced predominantly by natural killer cells (NK) and natural killer T cells (NKT) as part of the innate immune response, and by CD4 Th1 and CD8 cytotoxic T lymphocyte (CTL) effector T cells once antigen-specific immunity develops as part of the adaptive immune response.

As used herein, “OX-40” refers to TNF receptor superfamily member 4, also known as TNFRSF4, ACT35, CD134, IMD16 or TXGP1L. The protein encoded by this gene is a member of the TNF-receptor superfamily. This receptor has been shown to activate NF-kappaB through its interaction with adaptor proteins TRAF2 and TRAF5.

As used herein, “4-1BB” refers to TNF receptor superfamily member 9, also known as TNFRSF9, ILA, CD137, CD2137, or IMD109. The protein encoded by this gene is a member of the TNF-receptor superfamily. This receptor contributes to the clonal expansion, survival, and development of T cells. It can also induce proliferation in peripheral monocytes, enhance T cell apoptosis induced by TCR/CD3 triggered activation, and regulate CD28 co-stimulation to promote Th1 cell responses.

As used herein, “CD59” is also known as 1F5, EJ16, EJ30, EL32, G344, MIN1, MIN2, MIN3, MIRL, HRF20, MACIF, MEM43, MIC11, MSK21, 16.3A5, HRF-20, MAC-IP, or p18-20. This is a cell surface glycoprotein that regulates complement-mediated cell lysis, and it is involved in lymphocyte signal transduction. This protein is a potent inhibitor of the complement membrane attack complex, whereby it binds complement C8 and/or C9 during the assembly of this complex, thereby inhibiting the incorporation of multiple copies of C9 into the complex, which is necessary for osmolytic pore formation.

As used herein, “LAG-3” refers to lymphocyte-activation protein 3. The LAG-3 protein, which belongs to immunoglobulin (Ig) superfamily, comprises a 503-amino acid type I transmembrane protein with four extracellular Ig-like domains, designated D1 to D4.

As used herein, “TIM3” refers to hepatitis A virus cellular receptor 2, also known as CD366, KIM-3, SPTCL, TIMID3, Tim-3, TIMD-3, or HAVcr-2. The protein encoded by this gene belongs to the immunoglobulin superfamily, and TIM family of proteins.

As used herein, “IL-12R” is composed of interleukin 12 receptor beta 1 (IL-12Rβ1) and Interleukin 12 receptor beta 2 (IL-12Rβ2) chains, and mediates signal transduction, which involves the recruitment of Janus family tyrosine kinase 2 and signal transducer and activator of transcription (STAT)4.

As used herein, “CD28” refers to a protein encoded by this gene that is essential for T-cell proliferation and survival, cytokine production, and T-helper type-2 development. It is also known as Tp44.

As used herein, “CD57” refers to beta-1,3-glucuronyltransferase 1, also known as NK1, HNK1, LEU7, GLCATP or GLCUATP. The protein encoded by this gene is a member of the glucuronyltransferase gene family.

As used herein, “KIR” refers to killer cell immunoglobulin-like receptors, which are members of a group of regulatory molecules found on subsets of lymphoid cells.

As used herein, “KLRG-1” refers to killer cell lectin like receptor G1, also known as 2F1, MAFA, MAFA-L, CLEC15A, MAFA-2F1, or MAFA-LIKE. The protein encoded by this gene belongs to the killer cell lectin-like receptor (KLR) family, which is a group of transmembrane proteins preferentially expressed in NK cells.

As used herein, “CD27” refers to a member of the TNF-receptor superfamily. This receptor is required for generation and long-term maintenance of T cell immunity.

As used herein, “PD-1” refers to programmed cell death 1, also known as CD279, SLEB2, hPD-1, hPD-I, or hSLE1. Programmed cell death protein 1 (PDCD1) is an immune-inhibitory receptor expressed in activated T cells; it is involved in the regulation of T-cell functions, including those of effector CD8+ T cells.

As used herein, “CTLA-4” refers to cytotoxic T-lymphocytes associated protein 4, also known as CD, GSE, GRD4, ALPS5, CD152, IDDM12 or CELIAC3. This gene is a member of the immunoglobulin superfamily and encodes a protein which transmits an inhibitory signal to T cells. The protein contains a V domain, a transmembrane domain, and a cytoplasmic tail.

As used herein, “IL-2” refers to interleukin 2, also known as TCGF or lymphokine. This gene is a member of the interleukin 2 (IL2) cytokine subfamily which includes IL4, IL7, IL9, IL15, IL21, erythropoietin, and thrombopoietin. The protein encoded by this gene is a secreted cytokine produced by activated CD4+ and CD8+ T lymphocytes, that is important for the proliferation of T and B lymphocytes.

As used herein, “IL-10” refers to interleukin 10, also known as CSIF, TGIF, GVHDS, or IL10A. The protein encoded by this gene is a cytokine produced primarily by monocytes and to a lesser extent by lymphocytes.

As used herein, “TNF-α” refers to tumor necrosis factor alpha, which is a proinflammatory cytokine with an important role in the pathogenesis of several diseases.

As used herein, “microfluidic device” refers to a testing device that focuses on microfluidic behavior of fluids for precise control and manipulation in geometrically constrained small scale (typically sub-millimeter) at which surface forces dominate volumetric forces. A microfluidic chip is a pattern of microchannels, molded or engraved. This network of microchannels incorporated into the microfluidic chip is linked to the macro-environment by several holes of different dimensions hollowed out through the chip. It is through these pathways that fluids are injected into and evacuated from the microfluidic chip. Fluids are directed, mixed, separated or manipulated to attain multiplexing, automation, and high-throughput systems. The microchannels network design must be precisely elaborated to achieve the desired features. Microfluidics have diverse assets: faster reaction time, enhanced analytical sensitivity, enhanced temperature control, portability, easier automation and parallelization, integration of lab routines in one device.

As used herein, “conjugated” antibody refers to tagging on a protein, compound or dye in order to track its interaction with specific antigens. Fluorescent dyes such as Alex and DyLight fluor can be used in immunofluorescent assays. They can absorb and emit light at different wavelength for different labeling purposes. Antibodies conjugated with fluorescent dyes are used in immunoassays such as flow cytometry, ELISA, Western blot and fluorescence microscopy.

As used herein, the “simulation” or “activation” of T-cells refers to the binding of specific ligands to trigger biochemical signals in T-cells, including the production of interferon gamma.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| ELISA | enzyme-linked immunosorbent assay |
| ELISpot | Enzyme-linked immunosorbent spot |
| IFN-γ | Interferon gamma |
| IGRA | Interferon-gamma release assay |
| MERS | Middle East Respiratory Syndrome |
| PBMCs | Peripheral blood mononuclear cells |
| POC | Point of care |
| SARS-CoV-2 | Severe acute respiratory syndrome coronavirus 2 |
| CMV | Cytomegalovirus |
| RSV | Respiratory syncytial virus |
| HSV | herpes simplex virus |
| HBV | Hepatitis B virus |
| EBV | Epstein-Barr virus |
| TBI | Tuberculosis infection |
| TST | Tuberculin skin test |
| VOC | Variant of concern |

### BRIEF DESCRIPTION OF THE DRAWINGS

FIG. 1A. Schematics for standard and microfluidic chip ELISPOT assays.

FIG. 1B. IFN-γ independent evaluation of T-cells activation.

FIG. 1C. Thawed PBMC aliquots stimulated with or without PMA/ionomycin were cultured for 24 h in glass bottom wells coated with polysine, then incubated with a biotinylated secondary antibody, streptavidin-HRP and a chromogenic (red) HRP substrate or (Hoescht 33342 dye and a fluorescently tagged IFN-γ-specific antibody. White size bars indicate 75 μm.

FIG. 1D-E. PBMCs (~2×105) were seed on microplate wells coated with and without polylysine and stained with Hoechst 33342 to quantify the cell density of captured cells, or induced with PMA/ionomycin for 4 h, stained Hoechst 33342 and specific antibodies to IFN-γ, OX40, and 4-1BB, after which total cell numbers and activated T cell percentages were quantified using a fluorescent plate reader. Positive control (PC) wells were not washed to remove non- or weakly adherent cells.

FIG. 1F. One-Way two-sided parametric ANOVAs with Tukeys post-test were performed to analyze differences between the polylysine-coated and uncoated well values and (F) PMA-stimulated and unstimulated well values. Data indicate Mean±SD; *, p<0.05; **, p<0.01; ***, p<0.001; ****, p<0.0001; ns, no significant difference when analyzed by two-sided Mann-Whitney U-test.

FIG. 2A-C. T cell activation with SARS-CoV-2 spike peptide pool. Cryopreserved PBMCs from blood donors who received (A) 3 SARS-CoV-2 or (B-C) 0-3 vaccine doses were simulated by incubation with (A) PMA/ionomycin and/or (A-C) a SARS-CoV-2 peptide pool for up to 24 h, after which IFN-γ levels were evaluated by (A) ELISA, (B) Flow cytometry, or (C) ELISpot.

FIG. 2D-F. Freshly isolated PBMCs from unvaccinated and vaccinated (3 doses) donors were incubated with or without (D-E) a SARS-CoV-2 peptide pool, or (F) peptides derived from SARS-CoV-2, the M. tuberculosis (Mtb) proteins CFP-10 and ESAT-6 or HIV-1 p24 for 24 h, stained with Hoechst 33342, and incubated with IFN-γ-specific antibody. Total cell numbers and activated T-cell percentages were analyzed using a fluorescent plate reader Data indicate Mean±SD; *, p<0.05; **, p<0.01; ***, p<0.001; ****, p<0.0001; or ns, no significant difference between the indicated groups by (b) one-way parametric ANOVA with Tukeys post-test or (e-f) Mann-Whitney U-test.

FIG. 3A-F. Evaluation of on-chip ELISpot assay performance. Analysis the IFN-γ response to SARS-CoV-2 spike or HIV-1 p24 (non-specific control) peptides in PBMC samples (~2×106 cells) isolated from individuals without a history of HIV infection who had received three vaccine doses when analyzed by (A) our on-chip ELISpot, (B) flow cytometry, and (C) ELISpot assays. (D) Correlation of flow cytometry and on-chip ELISpot data. (E-F) On-chip ELISpot assays results from fingerstick whole blood samples (e) after pre-treatment with or without RBC buffer for one individual or (f) without RBC lysis for eight HIV-negative individuals more than six months after their second or third vaccine dose. t-test was performed to compare HIV-p24 or SARS-CoV-2 peptide pool stimulation. Data indicate Mean±SD;*, p<0.05; **, p<0.01; ns, no significant difference by two-sided Mann-Whitney U-test.

FIG. 4A-D. Activated T cell counting on glass surface. PBMC capture on well coated with and without different polylysine concentrations, seeded with 2×105 PBMCs, induced with PMA/ionomycin for 4 h, and stained with Hoechst 33342 and incubated with AlexaFluor488-(A), PE-tagged OX-40 (B) or APC tagged 4-1BB (C-D) specific antibodies, respectively. Wells were analyzed for activated T cell counts using a fluorescent plate reader. Positive control (PC) wells indicate signal detected in wells that were not washed to remove non- or weakly adherent cells.

FIG. 5A-C. PDMS microfluidic chip fabrication. (A) Silicon wafers coated with SU8 Epoxy are then covered with a photomask containing the device design, exposed to ultraviolet light, and washed with SU8 developer to remove inactivated SU8 surrounding the device design. Then a 10:1 PDMS-to-curing agent mixture is poured onto the master wafer and then heated at 60° C. for 5 h. (B) 50 μg/ml polylysine was coated on glass surface for 30 min and washed with DI water (C) Solidified PDMS device is cut from the wafer and bound to the polylysine coated glass slide previously exposed to oxygen plasma to generate the complete device.

FIG. 6A-B. Flow cytometry gating of blood cells samples. Example of the IFN-γ response detected upon flow cytometry analysis of ~2×106 PBMCs isolated from HIV-negative SARS-CoV-2-vaccinated (3 doses) after 24 h exposure to (A) SARS-CoV-2 spike peptides or (B) HIV-1 p24 (non-specific control) peptides. Scatterplots indicate the total PBMC scattering and lymphocyte gate (left panels) and the distribution of the IFN-γ-negative and IFN-γ-positive (gated population) in the lymphocytes gate (right panels).

FIG. 7A-E. Correlation of On-Chip IGRA results with traditional assays. (A-D) Correlations of on-chip ELISpot and flowcytometry results among the (A) SARS-Cov-2, (B) second and (C) third vaccine dose groups and (D) the vaccinated individuals with breakthrough infections. (E) Correlation of on-chip ELISpot assay and standard ELISpot assay results. Data indicate Spearmann/Pearson r-values.

FIG. 8A-K. Microchip response to T-cell capture.

FIG. 9A-G. Cumulative response of surface marker 4-1BB and OX-40

FIG. 10A-B. Comparison of IFN-γ with 4-1BB and OX40 response to TB infection.

FIG. 11A-H. Blood-based assay for enabling single-step diagnosis of TB infection.

### DETAILED DESCRIPTION

Enzyme-linked immunosorbent assay (ELISA) is a commonly used analytical biochemistry assay that uses a solid-phase type of enzyme immunoassay to detect the presence of a ligand (such as a protein) in a liquid sample using antibodies directed against the protein to be measured. The sample with an unknown amount of antigen is immobilized on a solid support, and after the antigen is immobilized, a detection antibody specific to the antigen is added to form a complex with the antigen. The detection antibody can be covalently linked to an enzyme or can itself be detected by a secondary antibody that is linked to an enzyme through bioconjugation. Between each step, the plate is typically washed with a mild detergent solution to remove any proteins or antibodies that are non-specifically bound. After the final wash step, the plate is developed by adding an enzymatic substrate to produce a visible signal, which indicates the quantity of antigen in the sample.

Enzyme-linked immune absorbent spot (ELISpot) is a type of assay that focuses on quantitatively measuring the frequency of cytokine secretion of a single cell. It utilizes antibodies to detect a protein analyte (the cytokine), much like ELISA. The mechanism of ELISpot starts with coating the wells with analyte-specific monoclonal antibodies. The second step is to incubate cells within the wells, during which the cells are allowed to react to any present stimuli and secrete the cytokine. Since the cells are surrounded by cytokine specific monoclonal antibodies that coat the walls of the wells, cytokines that has been secreted by the incubated cells will start to attach to the antibodies at a specific epitope. After washing the wells to remove unbound cells and undesirable substances, biotinylated cytokine-specific detection antibodies are then added to the well to bind to any cytokine that is left in the well, as the cytokine is still attached to the first set of antibodies used. Streptavidin-enzyme conjugate is then added to the wells to bind with the detection antibodies. The purpose of the cytokine-specific detection antibodies added to the wells in the previous step is so that the antibody can bind to the new streptavidin-enzyme conjugate, as the biotinylation creates a strong affinity between the biotin on the cytokine-specific antibody and the streptavidin on the conjugate. Finally a substrate is added to the wells, and is catalyzed by the enzyme conjugate to form insoluble precipitate that forms spots in the well. The spots can be read on an automated ELISpot reader or manually counted under a microscope.

Interferon-gamma (IFN-γ) is an important cytokine that is primarily produced by cells of the immune system, including innate-like lymphocytes, such as natural killer cells and innate lymphoid cells, and adaptive immune cells, such as Th1 cells and CD8+ cytotoxic T lymphocytes. Signals generated by the IFN-γ receptor activate the Janus kinase (JAK)-signal transducer and activator of transcription 1 (STAT1) pathway to induce expression of several genes that have necessary immune effector functions. IFN-γ also has critical roles in regulating the functions of specialized tissue cells, has effects on progenitor and stem cells, and participates in tissue and organ function under homeostatic, immune, and pathological conditions. Interferon-y release assays (IGRA) are medical tests used in the diagnosis of some infectious diseases. These tests are mostly developed for the field of tuberculosis diagnosis. For example, in patients with cutaneous adverse drug reactions, challenge of peripheral blood lymphocytes with the drug causing the reaction produced a positive test result for half of the drugs tested.

Currently there are two IFN-γ release assays available for the diagnosis of tuberculosis: QuantiFERON-TB Gold, as well as T-SPOT.TB. QuantiFERON-TB Gold (licensed in US, Europe and Japan) quantitates the amount of IFN-γ produced in response to the ESAT-6 and CFP-10 antigens from Mycobacterium tuberculosis, which are distinguishable from those present in BCG and most other non-tuberculous mycobacteria. T-SPOT.TB is a form of ELISpot, the variant of ELISA (licensed in Europe, US, Japan and China). It determines the total number of individual effector T cells expressing IFN-γ.

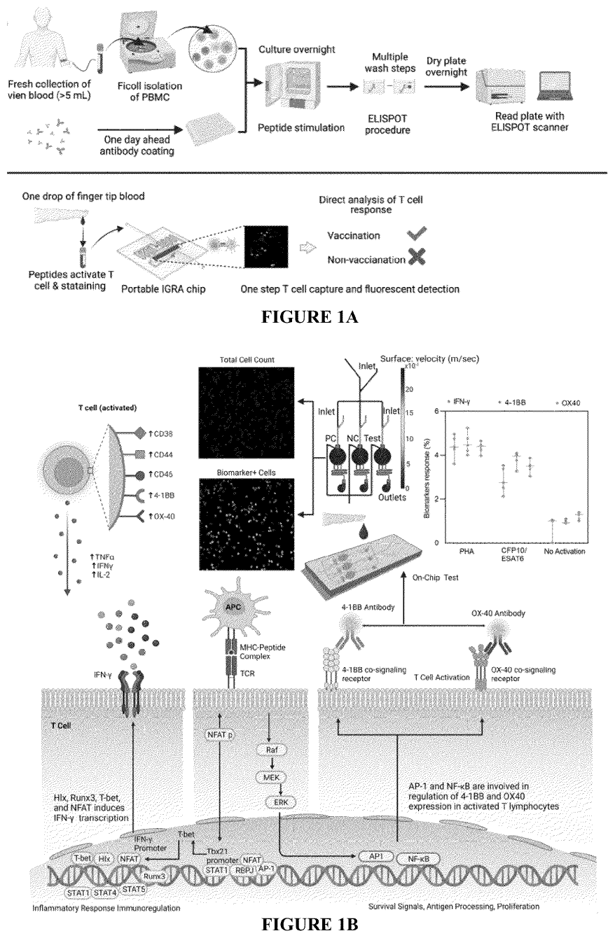

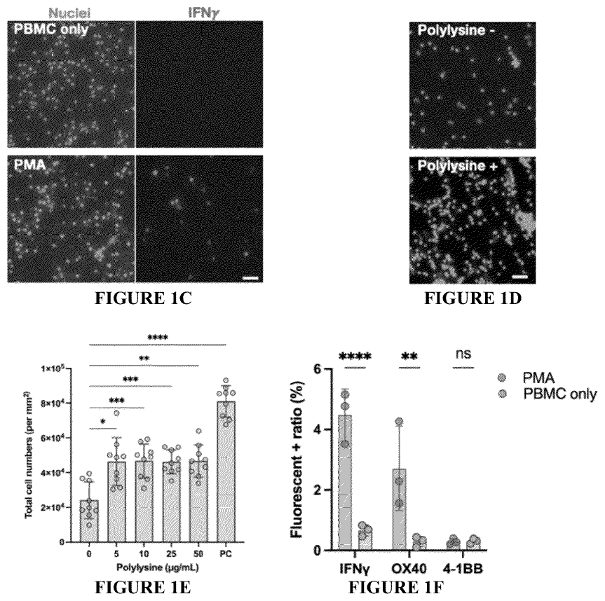

T cell activation requires extracellular stimulatory signals that are mainly mediated by T cell receptor (TCR) complexes. The TCR recognizes antigens on major histocompatibility complex molecules with the cooperation of CD4 or CD8 coreceptors. After recognition, TCR-induced signaling cascades that propagate signals via various molecules and second messengers are induced. Various signaling pathways, including the Ras-extracellular signal-related kinase (ERK)-activator protein (AP)-1 pathway, the inositol triphosphate (IP3)-Ca2+-nuclear factor of activated T cells (NFAT) pathway, the protein kinase C (PKC)θ-IκB kinase (IKK)-nuclear factor (NF)-κB pathway are involved in TCR signaling. Among which, NFAT pathway plays crucial role (FIG. 1B) in induction of IFN-γ from the activated T-cells.

Upon TCR activation, T-bet induces the production of IFN-γ and the activation of the transcription factors Hlx and Runx3 helps to induce STAT5, which additionally triggers IFN-y production. The release of IFN-γ from TB infected T-cells are conventionally evaluated using IGRA. However, IGRA fail to detect the full breath of T-cell response to any given antigen, as only a subgroup of T cells, including CD4 T helper cells are primarily responsible for the IFN-γ production. We sought to develop a platform, which can evaluate the activation response from entire T-cells repository to determine T-cells activation in an IFN-γ independent manner.

In search of IFN-γ independent T-cells evaluation, we started our study with the T-cells costimulatory signals (4-1BB and OX-40), also called T-cells surface markers (TSM), among others. Researchers showed that stimulation of the Ras GTP exchange molecule initiates a cascade of kinase activity involving Ras, MEK and ERK kinases (FIG. 1B), which lead to the activation of several transcription factors, including activating protein-1 (AP-1) and the transcription factor nuclear factor κB (NF-κB). These activated transcription factors regulate the transcription of a number of tumor necrosis factor (TNF) receptor superfamily genes, including 4-1BB and OX40. We deployed these TSM to develop a platform which can evaluate the full breadth of T-cells activation in IFN-γ independent pathway.

It is demonstrated that the usage of such platform for diagnosing tuberculosis infection (TBI) using a single step technique by collecting finger-stick amount of blood on a microfluidic chip and activating and staining the surface markers positive T-cells for fluorescence-based detection (FIG. 1B). We compared our method with intracellular IFN-γ staining for similar activation time and found that our approach had a similar or even better response in evaluation of T-cells activation. Compared to single cytokine, IFN-γ detection, multiplex T-cells surface marker expression analysis allows several additional avenues of hypothesis testing such as direct single-step detection of activated T-cells, comprehensive evaluation of T-cells activation from the entire T-cells repository, and frequency distribution of surface markers expression with minimal T-cells activation time.

The disclosure provides novel method for detecting T-cell specific interferon-y response by using only a small amount of blood sample and quick turnover time. Specifically, the method of this disclosure requires as little as 25 μL of whole blood sample without having to first isolate the peripheral blood mononuclear cells (PBMC). The method can provide results within 5 hours or less as opposed to conventional ELISpot that takes days to complete. The method utilizes microfluidic device to reduce the amount of sample required for detection.

The present invention is exemplified with respect to SARS-CoV-2 specific T-cell activation, as well as tuberculosis-specific T-cell activation. However, this is exemplary only, and the invention can be broadly applied to other pathogens that elicit T-cell activated interferon-y secretion. The following examples are intended to be illustrative only, and not unduly limit the scope of the appended claims.

Although examples were made with SARS-CoV-2 peptides, the inventors envision the device and method described herein are applicable to screening for other pathogens and/or antigens suitable for immunotherapies. Non-limiting examples include Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, and Trypanosoma cruzi.

Additionally, while the assay employed is ELISpot-based IGRA, other T-cell response evaluation can also be adopted. Non-limiting examples include CAR-T, TCR-T therapy, PD-1/PD-L1, CTLA-4, TIM3, LAG3 or other checkpoint blockage treatment in cancer. CAR-T (chimeric antigen receptor T-cell therapy) is a type of treatment in which a patient's T cells are modified in the laboratory to express chimeric antigen receptors such that they target cancer cells. TCR-T (T cell receptor-engineered T cell therapy) is similar to CAR-T, except the cells are modified to express T-cell receptors. PD-1 (programmed cell death protein-1) is an immune checkpoint on T-cells that keeps T-cells from attacking other cells (including cancer cells) in the body when it attaches to PD-L1, and by targeting PD-1 or PD-L1, the T-cells can be boosted to attack cancer cells. CTLA-4 (cytotoxic T-lymphocyte-associated protein-4), TIM-3 (T-cell immunoglobulin and mucin domain-3), and LAG3 (lymphocyte activation gene-3) are other immune checkpoints that show promise in cancer therapy.

### Example 1-Development and Optimization of a Microfluidic ELISpot IGRA System

ELISpot assays have greater procedural and equipment requirements than ELISA-based IGRAs, but are more readily adapted to a microfluidic assay workflow, since they can require fewer liquid handling steps in certain assay designs. The ELISpot microfluid workflow can be broken down into a few basic steps: blood collection, stimulation of T cells with pathogen-specific peptides, and the capture, staining, and analysis of activated T cells, most which can be accomplished on a microfluidic chip to greatly simplify the ELISpot workflow (FIG. 1(a)).

Since a microfluidic chips are routinely constructed on glass or plastic substrates, while ELISpot assays are usually performed on membranes that allow local capture of IFN-γ released by activated cells, we first evaluated our ability to detect discrete foci of a colorimetric substrate produced by activated T cells immobilized on a glass surface. This analysis found that it was possible to detect chromogen aggregation to identify the sites of IFN-γ expressing cells, but that the weak and diffuse nature of these signals did not permit accurate quantification of the number of activated cells (data not shown). However, analysis of intercellular IFN-γ expression in fixed and permeabilized PBMC samples by hybridization with an IFN-γ-specific fluorescent antibody revealed distinct foci of activated cells that were detected only in PBMCs samples pre-activated by with the phorbol myristate acetate (PMA) and ionomycin (FIG. 1C). PBMC binding was improved by precoating assay slides with polylysine (FIG. 1D), which is widely used to enhance cell capture, including lymphocyte capture, by forming electrostatic interactions with anionic molecules on their plasma membranes. A polylysine titration analysis found 5 μg/mL polylysine was sufficient to maximize mean PBMC adherence and capture (~4.5×10⁴ PBMC/mm²), doubling the cells captured on untreated slides (~2.2×10⁴ PBMC/mm²), to capture ~58% of the input PBMCs (~7.7×10⁴ PBMC/mm²) (FIG. 1E).

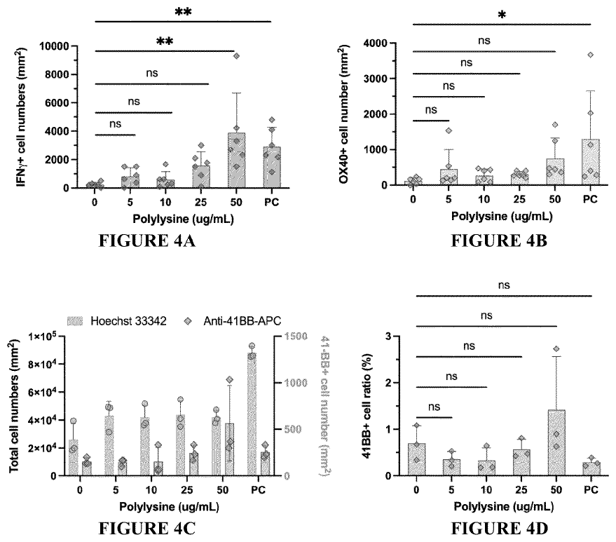

We next evaluated the ability of three biomarkers of T cell activation, intracellular IFN-γ, surface OX-40, and 4-1BB expression, to detect cell activation 24 h after captured PBMCs were stimulated with or without PMA/ionomycin. This analysis found similar low percentages of unstimulated PBMCs were positive for each marker, the percentage of 4-1BB positive cells did not increase after PMA/ionomycin stimulation (FIG. 4), and that the stimulated percentage of IFN-γ-positive cells was higher and less variable than the OX-40-positive cell values (FIG. 1F), leading us to select IFN-γ as the activation marker in all future experiments.

### Example 2-Detection of Antigen-Specific T Cell Activation Response on a Glass Surface

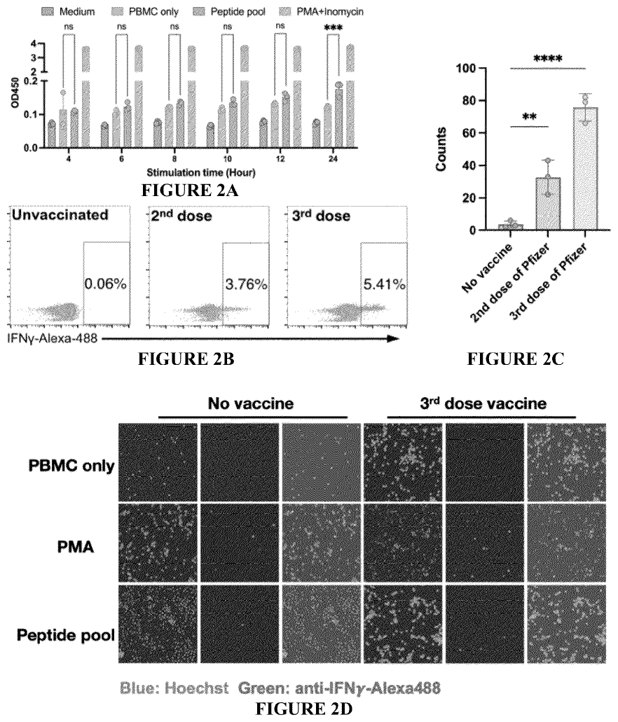

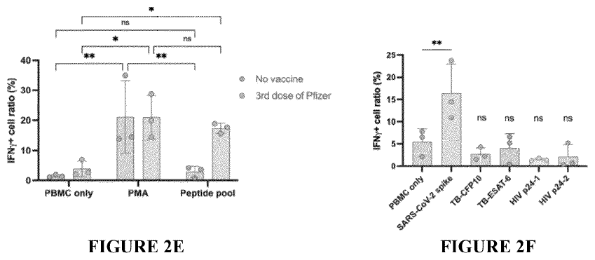

Extended incubation times required in standard ELISpot assays are a substantial issue for high throughput analyses. We therefore next evaluated the minimum time required to detect IFN-γ secreted by PBMCs isolated from an individual who had received three doses of a SARS-CoV-2 RNA vaccine. A standard ELISA detected maximum IFN-7 secretion by 4 h after PMA/ionomycin stimulation, although significant IFN-γ secretion was not detected until 24 h after stimulation with a pool of SARS-CoV-2-derived peptides (FIG. 2A). A subsequent flow cytometry analysis performed to measure the fraction of SARS-CoV-2 responsive T cells present in PBMC fractions of unvaccinated and vaccinated individuals (FIG. 2B), detected a low rate of responsive cells in the unvaccinated PBMC sample after 24 hour incubation (0.01%), which progressively increased in individuals who received two and three vaccine doses (3.76% and 5.41%, respectively). These differences were more obvious when these samples were analyzed using a standard ELISpot IGRA, where the PBMC response rate for unvaccinated individuals was only about 5 secreted IFN-7 clusters compared to 36 and 77 clusters for individuals who received two or three vaccine doses (FIG. 2C).

Given that our microfluidic ELISpot assay format employs fluorescent antibodies to detect intracellular IFN-γ expression, we next evaluated its ability to detect cell activation rates in a PBMC isolated from unvaccinated and fully vaccinated individuals (3 vaccine doses) 24 h after incubation with a pool of SARS-CoV-2-derived peptides or PMA/ionomycin. This analysis determined that the cell activation rates in PBMCs of fully vaccinated (Pfizer) versus unvaccinated/unexposed individuals were ~20% and ~5%, respectively, whereas PMA/ionomycin tended to activate ~20% of the PBMCs in both groups (FIG. 2D-E). Notably, the frequency of IFN-γ-positive cells in the unvaccinated group was substantially higher (20% compared to 0.01%) than in the flow cytometry analysis, which used a similar intracellular IFN-γ staining procedure. This difference suggests either differences in the samples analyzed in these studies, or differences in the sensitivity, activation efficacy, or analytical sensitivity of these two procedures. However, the frequency of IFN-γ-positive PBMCs detected in this analysis was found to be pathogen specific, since PMBC activation frequencies detected when these cells were incubated with peptides from other human pathogens to which their donors had not been exposed or vaccinated (e.g. TB and HIV) were not different from those measured with unstimulated PBMCs (FIG. 2E).

### Example 3-Microfluidic Chip Elispot IGRA Performance

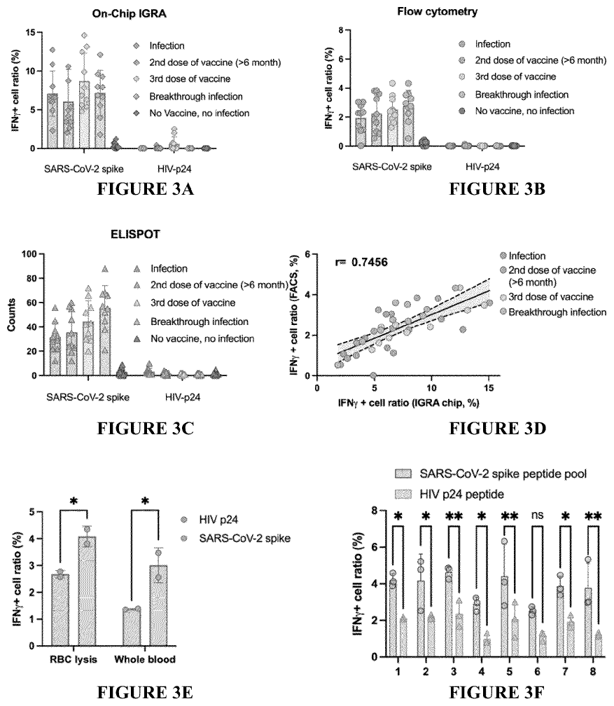

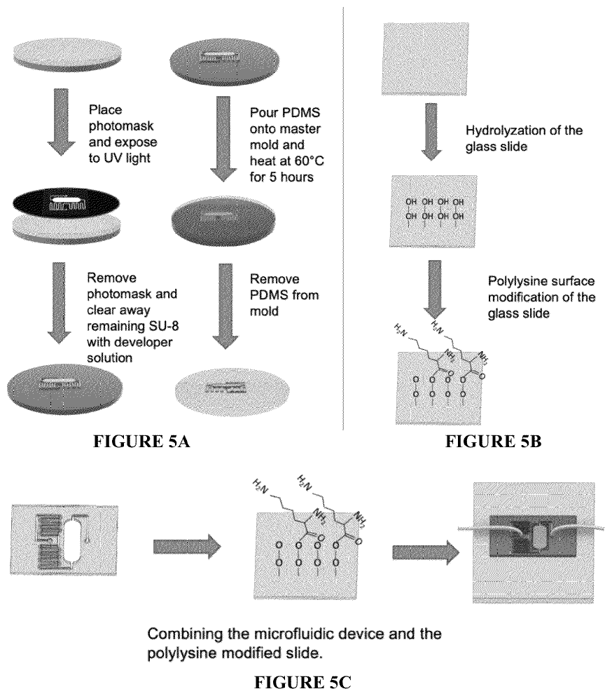

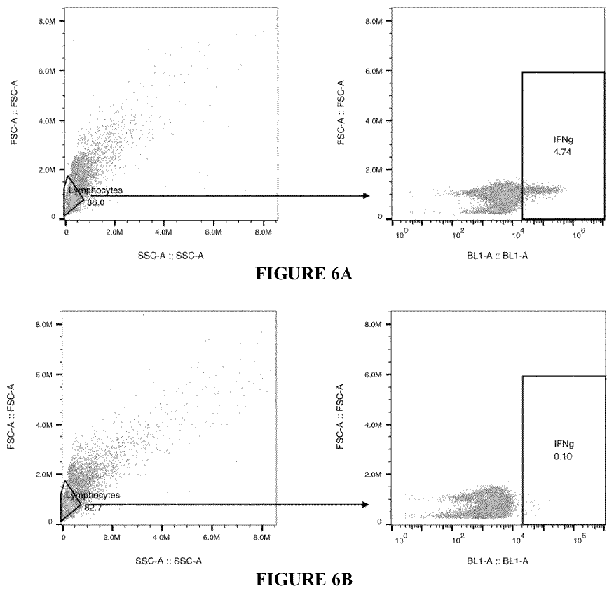

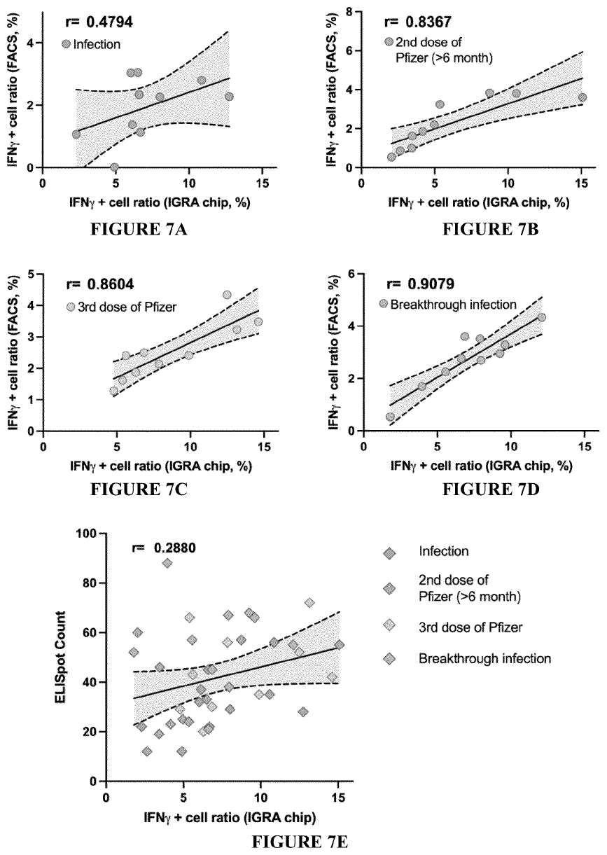

These cell capture, stimulation, and analysis steps were then combined into an integrated microfluidic assay procedure that was used to evaluate on-chip assay performance. Microfluidic chip (FIG. 5) wells were loaded with 2×10⁶ PBMCs (data not shown), which were captured on a polylysine layer and then cultured in peptide-spiked culture media for 24 h, fixed, permeabilized and incubated with Hoescht 33342 and an IFN-γ-specific fluorescent antibody for 20 min, after which on-chip fluorescent microscope images of labeled cells were analyzed to evaluate PBMC activation. Cell activation backgrounds detected for the unstimulated PBMC samples in this on-chip analysis were similar markedly lower than previously detected, as was the percentage of cells stimulated upon incubation with the SARS-CoV-2 peptide pool, which did not differ among the vaccinated and/or infected groups (FIG. 3A). ELISpot and flow cytometry analyses (FIGS. 3B & 6) of these samples produced similar results, but revealed modest progressive increases in with groups with based on the number antigen exposure events (FIG. 3B-C). Notably, the mean percentage of IFN-γ-positive cells detected in all groups with the on-chip assay (5.3±4.2%) was higher than flow cytometry (1.8±1.3%), which also analyzed intracellular IFN-γ expression, although both data sets revealed good correlation, which was much less pronounced with standard ELISpot data (FIGS. 3D and 7). And flow cytometry and chip read are most consistent for the 3 vaccine dose group and least consistent with the infected group (FIG. 7)

### Example 4-Microfluidic IGRA Analysis of Fingerstick Whole Blood Samples

ELISpot assays isolate and culture PBMCs from >5 mL of venous blood, which renders them unsuitable for POC tests or use in resource limited settings. We therefore evaluated whether our ELISpot assay, could be performed with fingerstick blood draw volumes (~25 μL) with or without a red blood cell (RBC) removal step using a 4 h peptide incubation step. RBC lysis increased the number of captured PBMCs versus whole blood samples, but also increased the IFN-y response to a control peptide, resulting in a corresponding decrease specific to control peptide induction ratio (2.2-fold versus 1.25-fold) in these samples (FIG. 3E). Subsequent analysis of fingerstick whole blood samples collected from individuals with no history of HIV infection more than six months after their third vaccine dose detected a similar degree of specific induction in all samples (2.4±0.8 fold induction), which reached significance in all but one sample (FIG. 3F). The mean IFN-γ-positive cell percentage detected in this analysis (3.8%) was lower than observed in on-chip ELISpot assays performed with isolated PBMC samples from other vaccinated individuals (9.6%), however this was balanced by reduced sample variance.

Immunoassays that respectively detect the presence or titer of specific antibodies to pathogen-derived factors and the percentage and activity of T cells that respond to these factors provide important, but divergent, information that is useful in evaluating the efficacy of an individual's potential immune response. Specific antibody assays are straightforward and can be readily employed in most settings, and are thus often suitable for use as POC tests, but may not provide a reliable picture of immunity as circulating antibody responses can wane long before the loss of inducible immunity. IGRAs are potentially useful to address this question, but are not suitable for high-throughput use or use in resource limited settings and thus are not practical for the evaluation of individual immune response at large scale. Here we demonstrate that a modified ELISpot IGRA can be employed to address the shortcoming of traditional ELISAs, since it can be performed using fingerstick rather than venous blood volumes; analyze whole blood, eliminating the need for sample processing to isolate PBMCs; and be read within ~5 hours of sample collection using a fluorescent microscope or plate reader.

An ELISpot assay approach was chosen for this analysis since this assay format measures the fraction of T-cell that are responsive to a selected pathogen-derived factor, and thus provides a direct measure of the cell population available to respond to this pathogen. ELISA-based IGRAs, which are more commonly used, measure the relative degree of the cytokine response, and thus integrate the number of available cells and the extent of their inducible cytokine response. ELISpot and ELISA-based IGRA do not exhibit strong correlation, unlike ELISpot and flow cytometry assay data, which demonstrates good correlation, albeit with substantial variation.

Standard ELISpot assays detect the number of cells secreting a factor and thus require this factor be bound at the site of its release for subsequent detection in order to estimate the number of signal-positive cells in a known number of input cells, are problematic for use at high-throughput in low resources settings.

Conventional ELISpot assays analyze dye foci deposited on polyvinylidene difluoride (PVDF) membranes, but this readout approach employs a sandwich ELISA in which IFN-γ released by activated cells is captured on the PVDF membrane at their position and hybridized with an enzyme-conjugated IFN-γ-specific antibody to permit in situ conversion and binding of a colorimetric substrate. This requires multiple wash steps, careful control of incubation and reaction times, and a readout device that can capture high magnification images illuminated with a high intensity light source, complicating the assay workflow, increasing equipment demands, and reducing utility in resource limited settings. Replacing this approach with an equilibrium-based intracellular staining workflow used in flow cytometry simplifies and reduces the stringency of its readout procedure, which employs the co-detection of nuclear staining to visualize relative cell activation rates without requiring the analysis of predetermined numbers of PBMCs. RBC removal was not required for this analysis since this process did not markedly reduce PBMC binding and appeared to increase non-specific cell activation thereby Decreasing the relative degree of specific induction. However, a nuclear RBCs were not included in the cell count and thus did not affect calculated cell activation percentages.

Standard ELISpots assays also evaluate the number of IFN-γ-positive cells within a known and standard concentration of viable PBMCs requiring isolation of the cells, determination of cell viability, and extended culturing at consistent amounts. All of these requirements add complexity that renders these assays impractical for use in many analysis settings. However, our revised ELISpot assay employs fingerstick whole blood microsamples, eliminating the need for a trained phlebotomist to perform a venous blood draw and then need to isolate PMBCs, while the number of IFN-γ-positive and total PBMCs present in analysis sample can be directly detected from captured assay images. Given the limited opportunity for variation in the sample collection and processing procedure, it can also be assumed that cell viability should not influence the IFN-γ-positive cell percentages in this approach.

Although this rapid streamlined ELISpot assay eliminates most obstacles that limit the widespread use of IGRAs, several aspects could be further optimized to improve assay performance. For example, the assay does not distinguish between groups of individuals who have different exposure histories to the targeted antigen through infection and/or been infection, which can be detected with standard assays. This may be due to the relatively small number of cells captured on the microwell, the loss of activated T cells during the washing step, and/or sampling bias during image capture and analysis. Improving the precision and reproducibility of such assay measurements may be important to improve the ability to sensitively track the durability of acquired T cell responses to specific pathogen-derived antigens and the relative amount of protective immunity retained with the passage of time. Enhanced precision could be obtained using several approaches, either alone or in combination. Cell capture was enhanced by a polylysine coating on the assay well surface, but this electrostatic interaction approach is not T cell-specific and may limit the ability to reproducibly capture and retain T cells from whole blood during the assay procedure. Conjugation of the assay well surface with CD4- and/or CD8-specific antibodies could improve the number and surface retention of T cells captured from off-chip cell inducing and staining reactions to reduce assay variability. This approach might increase cell clustering events that potentially obscure the number of total and/or positive cells present in an assay. However, the impact of clustering could be offset by fabricating assay chips so that antibodies are spotted in a dispersed array. Finally, an ideal version of this ELISpot assay would be read by an inexpensive portable device to allow analysis on-site in resource limited settings. We have previously developed an inexpensive fluorescent smartphone microscope and app that can be used to read other chip-based assays, and which could be modified to read, analyze, and report results for our current ELISpot assay. This technology could also allow recovery of activated cells for single-cell intracellular or secreted protein analyses with minimal modifications.

In summary, this ELISpot serves as a platform to rapidly and inexpensively analyze T cell responses to specific antigens using fingerstick whole blood microsamples, without requiring significant equipment or technical expertise. This platform should allow high-throughput analysis of T cell responses to specific pathogen derived antigens as a measure of potential immunity following previous exposure via infection or vaccination. For example, the potential resistance to new variants of these pathogens can be evaluated by modify SARS-CoV-2 peptide pool by adding specific mutated peptides. Alternately, a variant of this approach could also be adapted to measure memory B cell responses. This capacity should permit large-scale evaluation of acquired immune responses to benefit the evaluation of vaccine effectiveness for existing and emerging infectious diseases and may also facilitate improved understanding of some chronic infections.

### Example 5 T-Cell Enrichment and Activation Time in Diagnosis Outcomes

To develop a clinically useful single-step whole blood assay for tuberculosis infection, multiple prerequisites are considered. We needed to define an entire T-cells population by a universal labeling approach that covers an expanded population of TB patients. We also needed to reduce background signals and specifically detect recently activated T-cells response. We evaluated our assay to detect discrete foci of a colorimetric substrate produced by activated T-cells immobilized on a glass surface. However, the weak and diffuse nature of these signals did not permit accurate quantification of the number of activated cells. Therefore, we optimized a labeling approach using fluorochrome Hoescht 33342 to fluorescently label the nuclear DNA of all T-cells. This approach has no anticipated bias toward different cell types and allows us to calculate the fraction of surface marker-positive T-cells and identify staining artifacts in non-Hoechst labeled objects.

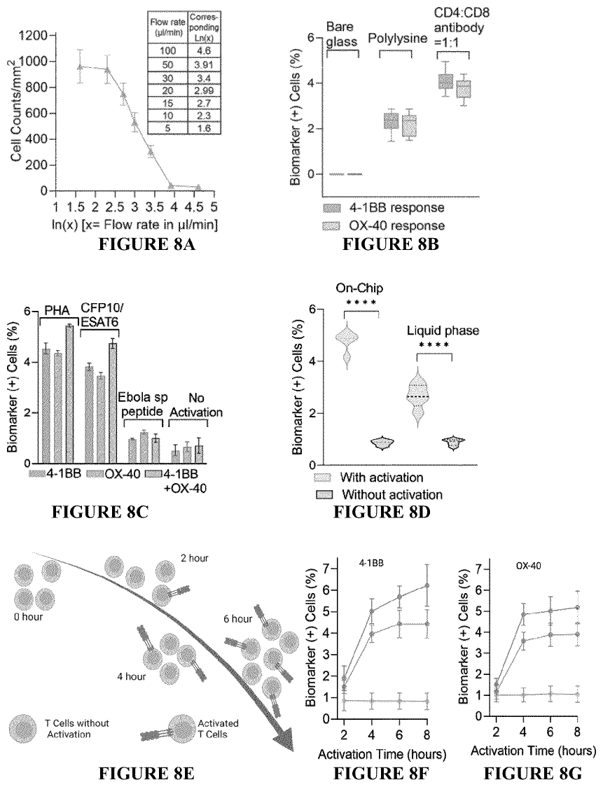

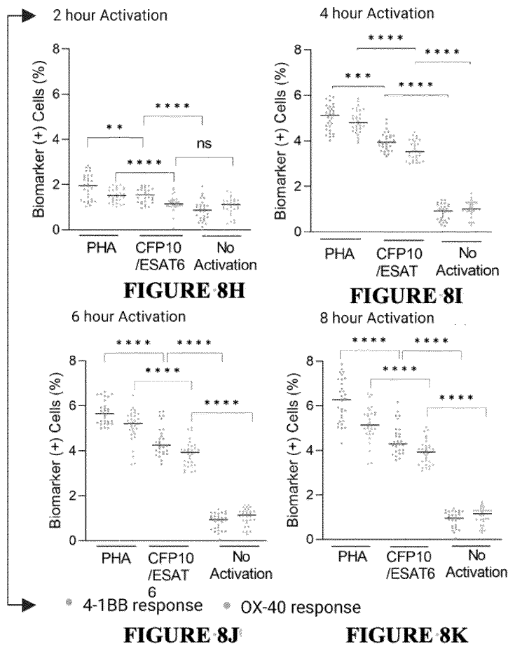

To maximize T-cell counts on the reaction chamber, we studied the effect of flow rate on T-cell attachment inside the chip (FIG. 8A). Successful firm adhesion of target cells in the bio-functional reaction chamber depends on the formation of bonds that hold cells against hydrodynamic loadings and shear stresses that induce bond dissociation. Continuous exposure to stress can affect T-cell shape, proliferation, migration, or protein expression, affecting their ultimate function. Thus, controlling the flow rate during cell introduction and following buffers is critical. High flow rates result in a small number of captured cells, favoring detachment and rolling over the surface, while low flow rates lead to the capture of many normal cells along with the desired cells. We optimized the flow rate at 10 μl/min to balance the shear force and binding force and maximize specific binding of target cells. We labeled all T-cells captured on the reaction chamber with Hoescht 33342 dye for identification purposes. We evaluated different flow rates (5 μl/min, 10 μl/min, 20 μl/min, 60 μl/min, 95 μl/min, and 120 μl/min) and found that the cell count in the reaction chamber significantly decreased above a flow rate of 20 μl/min, while a flow rate below 5 μl/min led to nonspecific cell attachment.

Next, we optimized the surface chemistry for the maximum T-cells response from the microchip. Polylysine coating on the microfluidic chip captured T-cells along with other peripheral blood cells, masking the T-cell-specific signals in the assay. Other approaches, such as immobilization of T-cell-specific antibodies on the chip using glutaraldehyde, require additional blocking steps to reduce nonspecific bindings of peripheral cells to the aldehyde sites. Glutaraldehyde itself causes autofluorescence signals, increasing the background noise of the detection assay. Therefore, we optimized the on-chip T-cell capture approach using CD4 and CD8-specific antibodies via EDC-NHS chemistry. The biomarkers positive cells percentage was found to be higher when CD4 and CD8 T-cells specific antibodies were immobilized on the chip surface than nonspecific attachment of all PBMCs with polylysine coating (FIG. 8B).

To ensure the specificity of our sensor platform, we conducted positive and negative control experiments on 10 T-SPOT.TB positive PBMC samples, along with T-cell activation by TB-specific antigens (FIG. 8C). Phytohemagglutinin (PHA) was used as a positive control to stimulate TSPOT.TB positive PBMC samples, and the response without T-cell activation was considered a negative control. The TSPOT.TB positive PBMCs were stimulated with an Ebola-specific peptide to evaluate nonspecific responses. The nonspecific stimulation showed a similar response to the negative control, validating the specificity of our sensor platform.

Finally, we compared the T-cell response obtained from the optimized microfluidic chip vs the liquid phase detection of T-cell response directly from PBMCs. The response obtained upon T-cell activation was significantly higher using microfluidic chip than liquid phase-based detection (FIG. 8D). These findings demonstrate that the high specificity of receptor-ligand binding provides an extremely sensitive means for T-cell manipulation, selection, and T-cell-based diagnostics.

Next, we aimed to determine the minimum activation time required to elicit surface marker expression (specifically 4-1BB and OX-40) on T-cells (FIG. 8E). The anti-4-1BB antibody used herein is Cd137 (4-1BB) Monoclonal Antibody (4B4 (4B4-1)), FITC, eBioscience 11-1379-42. The anti-OX-40 antibody used herein is Cd134 (OX40) Monoclonal Antibody (ACT35 (ACT-35)), FITC, eBioscience 11-1347-42.

We conducted this study on a cohort of 30 TSPOT.TB positive patient's PBMC samples using different activation times (2, 4, 6, and 8 hours) with TB-specific peptide antigen (CFP-10/ESAT-6) stimulation. To ensure specificity, we also included positive control (PHA stimulation) and negative control (no stimulation) experiments (FIG. 8E-8K). Our results showed the elicited response of T cells activation markers (4-1BB and OX-40) for 2-hour activation duration may not be sufficient to achieve significant change of response of 4-1BB and OX-40 induced by CFP10/ESAT6 stimulation compared to negative control base line value (FIG. 8F-8G). However, with more stimulation time (4h, 6 h, 8h) (FIG. 8H-8K) more T-cells became activated and expressed surface markers on their surface. The expression of surface markers dynamically increased with time starting from 2-hour activation time. At 4-hour activation time, T-cells provided sharp change of response compared to negative control, differentiating them from the base signal (the negative control response). Therefore, we chose a 4-to-6-hour T-cell activation time for further clinical testing.

### Example 6 Cumulative Response of the Surface Immune Markers 4-1BB and/or OX-40

The efficacy of the newly developed platform was tested in diagnosing tuberculosis infection first in a HIV negative PBMC clinical cohort. To do so, T-cells were enriched on the microchip surface and evaluated the response of surface markers after 6 hours of stimulation. A total of 20 TSPOT.TB positive and 20 TSPOT.TB negative PBMC samples obtained from Houston Methodist were tested, using TB specific peptides including CFP-10 and ESAT-6 to activate T-cells. We tested the intracellular IFN-γ, surface expression of 4-1BB, and OX-40 individually using the platform.

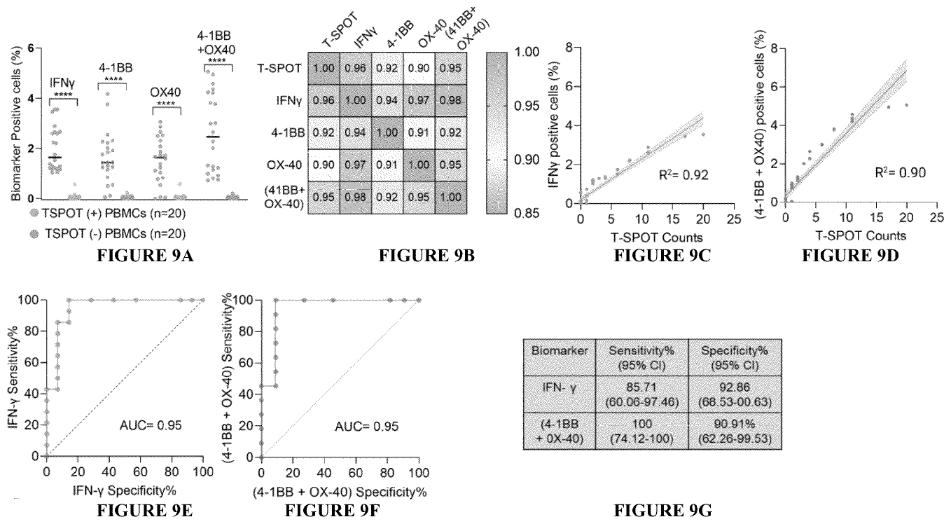

Next, the cumulative response of 4-1BB and OX-40 expression together was evaluated by adding a cocktail of fluorescent-tagged antibodies for each of the biomarkers tested using on-chip technology. As shown in FIG. 9A, we observed an overlap between the individual 4-1BB and OX-40 signals of the TSPOT+ and TSPOT− groups, whereas we found distinct signals and no overlap of intracellular IFN-g and cumulative 4-1BB+OX40 signals from TSPOT+ and TSPOT− groups. Based on this observation, we proceed with testing of cumulative response of 4-1BB and OX-40 for future samples, instead of their individual response.

The direction of the linear relationship between the biomarker's response (intracellular IFN-γ, 4-1BB and OX-40) was also evaluated with respect to TSPOT outcomes. The covariance matrix (FIG. 9B) indicates intracellular IFN-γ and cumulative response of (4-1BB+OX40) are more closely correlated with TSPOT counts (with Pearson's R coefficient 0.96 and 0.95 respectively) than the individual surface markers 4-1BB or OX40 response, which further support the fact that cumulative response of (4-1BB+OX-40) could be a potential strategy to diagnose the TB infection.

The intracellular IFN-γ and the cumulative response of TSM (4-1BB and OX-40) showed a strong correlation (r2 value 0.92 and 0.90, respectively) with the TSPOT.TB outcomes of the clinical cohort (FIG. 9C-D). We further analyzed the data using Receiver Operator Characteristics (ROC) regression modeling to retrospectively measure the overall predictive performance of our assay (FIG. 9E-F). The area under the ROC curve (AUC) is a measure of the accuracy, or the discriminative capacity of the assay, and ROC curve analyses show the tradeoff between the differences in sensitivity and specificity of tests performed in the assay.

The instant assay demonstrated high specificity (~91% with 95% CI) for cumulative surface markers (4-1BB+OX-40) response, with sensitivity being significantly higher (100% with 95% CI) for the cumulative surface markers response than for intracellular IFN-γ (85.71% with 95% CI) (FIG. 9G). Moreover, the data indicated that a single-step evaluation of cumulative surface markers expression on activated T-cells surface was sufficient for diagnosing tuberculosis infection. This eliminates the need for individual TSM (41BB or OX40) response analysis and fixation or permeabilization steps for intracellular cytokines detection. In summary, this newly developed on-chip platform has the potential to revolutionize tuberculosis infection diagnosis, with high sensitivity and specificity for single-step point of care detection.

### Example 7: Evaluation of TB Cohorts of Immunocompromised Patients Using On-Chip Assay

Next, the response of the instant microchip assay was evaluated for the immunocompromised patient cohort. A total of 37 samples were collected between 2021 to 2023 in Texas Children's Hospital. Among all the samples, 16 samples were from patients and the remaining 11 were from healthy individuals. We performed a blind test for this cohort of samples using our microchip assay and evaluated the microchip outcomes with respect to the patient's clinical diagnosis, sputum culture, Gene Xpert and IGRA outcomes. Each sample collected were divided in to four categories for testing, including positive control (with PHA stimulation), negative control (without activation), microchip outcomes for cumulative (4-1BB+OX40) response with (CFP10/ESAT6) peptide stimulation and intracellular IFN-g response with (CFP10/ESAT6) peptide stimulation.

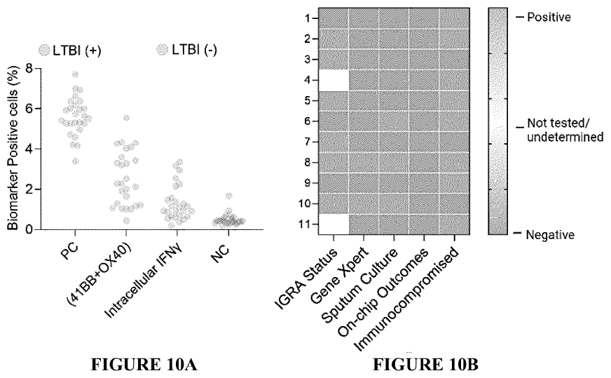

The results showed that the intracellular IFN-g response was not clearly distinguishable for LTBI positive and LTBI negative groups. However, a clear distinction of the (41BB+OX40) response was observed between LTBI positive and LTBI negative group (FIG. 10A). This further supports our study demonstrating the advantage of using cumulative response of (4-1BB+OX40) as the biomarker for LTBI diagnosis. Among the 11 patients' samples clinically tested for LTBI, a total of 4 samples showed IGRA status negative but clinical diagnosis, gene Xpert and sputum culture positive, indicating limited gain of IGRA test for these 4 patients (FIG. 10B). The microchip test outcomes for these patients found to be TBI positive demonstrating the benefits of cumulative (4-1BB+OX-40) response for the patient groups who did not respond well in the IGRA test. These 4 patients were further diagnosed with pulmonary pleural coccidioidomycosis, pulmonary cavitary lesion, strongyloidiasis and primary immunodeficiency respectively. The overall outcomes show the potential of cumulative (4-1BB+OX-40) based diagnosis for the patients who have limited gain from IGRA.

### Example 8: Blood-Based Assay for Single-Step Diagnosis of Tb Infection

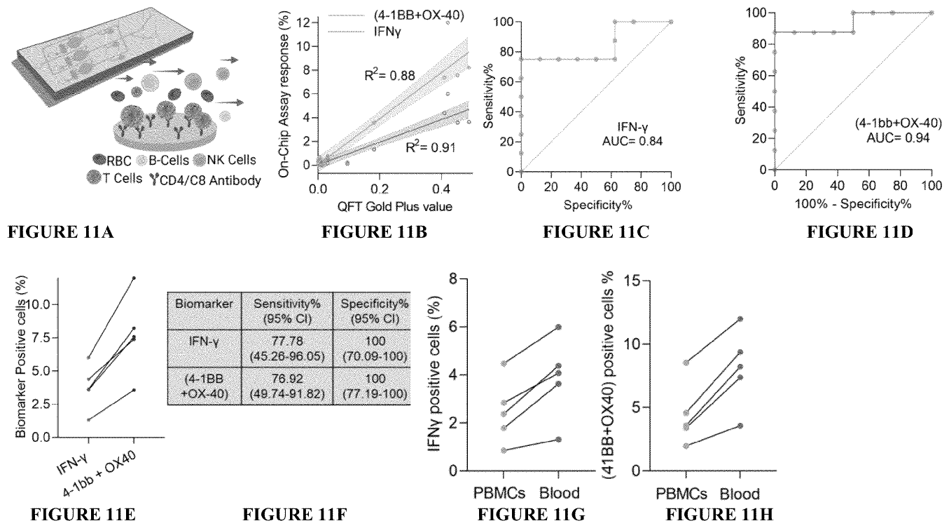

The conventional PBMC based assays require extended culture of PBMCs isolated from large volumes of venous blood, making it difficult to use them in resource-limited settings. To address this issue, the instant microchip assay was tested directly from fingerstick blood volumes (approximately 25 μL) without the need for an intermediate red blood cell lysis step (FIG. 11A). This assay uses antibodies specific to CD4 and CD8 T-cells to capture these cells on the sensor surface while removing other components of the blood through washing steps. We conducted clinical testing on a cohort of 20 patients using our assay in a blind manner. The blood samples were obtained from Ochsner Health, and their clinical information and QuantiFERON (QFT) gold plus assay outcomes were initially unknown to us. The intracellular IFN-γ and the cumulative response of 4-1BB and OX-40 showed a strong correlation (r2 value 0.91 and 0.88, respectively) with the (QFT) gold plus outcomes of the clinical cohort (FIG. 11B).

We further analyzed the data using Receiver Operator Characteristics (ROC) regression modeling to retrospectively measure the overall predictive performance of this assay (FIGS. 11C and 11D). The area under the ROC curve (AUC) value found to be 0.84 for intracellular IFN-γ and 0.94 for cumulative (4-1BB and OX40) indicating better diagnostic discriminatory ability of cumulative (4-1BB+OX40) response than the intracellular IFN-γ for TBI diagnosis. Further, we observed significantly higher response rate from cumulative (41BB+OX40) than intracellular IFN-γ for the QFT gold plus positive samples (FIG. 11E).

The ROC regression modeling measured the overall predictive performance of this microchip assay for testing from direct blood sample and we found that the sensitivity of the blood-based assay for intracellular IFN-γ and cumulative surface markers response (4-1BB+OX40) was similar, at around 78% and 77%, respectively and specificity 100% for both (FIG. 11F). Importantly, direct blood-based analysis provided higher specificity (100% with CI 95%) for intracellular IFN-γ and cumulative surface markers response than the PBMC-based assay (FIG. 11G). To determine the whole blood vs. PBMCs response rate from the same sample cohort, QFT gold plus TBI positive samples from this cohort were selected and compared the response we achieved from blood samples and the isolated PBMCs of the respective blood samples. We observed a significant increase in response rate in the whole blood samples compared to the isolated PBMCs for both the intracellular IFN-γ and cumulative (41BB+OX40). This microchip assay showed approximately 2% increase in response rate of intracellular IFN-γ for whole blood sample compared to PBMC samples (FIG. 11G). Whereas for cumulative response of (41BB+OX40), the microchip assay showed almost 4-5% increased response for blood-based assay compared to PBMCs based assay (FIG. 11H). In summary, we were able to successfully use direct blood sample without RBC lysis step for our microchip assay and made the TBI diagnosis one-step, fast, ultrasensitive, and feasible for point-of-care diagnostics.

It is shown that IGRA fail to detect the full breath of a T-cell response to any given antigen, as only a subgroup of T cells, including CD4 T helper cells are primarily responsible for the IFN-γ production. The latent TB infected individuals with low peripheral lymphocyte (CD4 T cells) counts, immunocompromised, or suffering from extra pulmonary TB infection have limited gain from IGRA. In addition. the patient population in their active stage of TB infection cannot gain benefits from the IGRA because of the sharp increase of regulatory T (Treg) cells in the active TB infection state suppress the IFN-γ release from the helper T(Th) cells. Decreased frequencies of M. tuberculosis specific CD4 T-cells producing Th1, Th2 and Th17 cytokines, including IFN-y, is the primary reason the individuals with HIV have limited gain from IGRA.

Additionally, a significant clinical gap still exists in TB infection screening among HIV infected patient groups, increasing overall TB incidence and associated mortality. Further, the common issues observed for TST and IGRA includes, maintenance and processing of samples, requirement of 2-10 ml of blood (limiting the usage in very young children), prolonged diagnosis (Sample-to-result interval is more than 24 hours), errors in collecting or transporting blood specimens, which further affect the accuracy of the diagnosis. Reduced access to TB diagnosis and treatment are one of the primary reasons for TB related deaths. To address these issues with the existing technology for TBI diagnosis, we started our research to develop an IFN-T independent point-of-care technology.

### Method

Patient population: Whole venous blood and fingerstick blood samples were obtained from a population of SARS-COV-2-infected and/or vaccinated adults enrolled in our study at New Orleans Children's Hospital.

Subjects or households with suspected or confirmed SARS-CoV-2 infection were recruited from the Greater New Orleans community under Tulane Biomedical Institutional Review Board (federal-wide assurance number FWA00002055, under study number 2020-585). Enrolled subjects completed a study questionnaire regarding infection and demographic information and provided a blood sample.

For the fingerstick blood analysis studies, healthy SARS-COV-2-vaccinated adults aged 21 to 41 years were enrolled in the study in accordance with an protocol approved by the Institutional Review Board of Tulane University. Written informed consent was obtained from each participant before study participation. A SARS-SOV-2 screening questionnaire and information regarding vaccination status were also obtained. Fingertip blood samples were collected from each participant using a contact-activated lancet (BD 355594) to collect 400-800 μL of blood into lithium heparin micro blood collection tubes (BD 365965) which were then immediately processed for on-chip ELISpot analysis. No adverse reactions or infectious sequalae were reported by participants.

PBMC isolation: PBMCs were isolated from frozen leukophoresis samples (Stemcell Technologies) or whole blood samples. Venous blood samples were collected in EDTA tubes and supplemented with a 15× volume of cold (4° C.) isotonic ammonium chloride solution, mixed by inversion at room temperature for 10 minutes using a rotary mixer set to ~500 rpm to allow RBC lysis, and then centrifuged at 250 g for 10 minutes. Cell pellets were then resuspended in 1 mL PBS, and this cell suspension was layered over 10 mL of Ficoll-Paque PLUS Media (Cytiva 17144002) in a 15 mL centrifuge tube centrifuge at 500 g for 20 minutes in a swinging buck rotor to isolate PBMCs following the manufacturer's instructions. Isolated PBMCs were resuspended in 5 mL AIM V cell culture media (Fisher Scientific 31-035-025), aliquots were analyzed to determine viable cell concentrations by staining cells with a 0.4% Trypan Blue solution, and cells suspensions adjusted to a final concentration of 3×10⁶/mL in AIM V cell culture media (Fisher Scientific 31-035-025), mixed with 40% fetal bovine serum and 20% dimethyl sulfoxide, and then stored in the vapor phase of a liquid nitrogen dewar.

Blood was collected from subjects and plasma and peripheral blood mononuclear cells (PBMCs) and were isolated by density gradient centrifugation in Leukosep tubes (Greiner Bio One) and Ficol-Paque PREMIUM 1.078 μg/ml (Cytiva). PBMC were washed, counted, and suspended in FBS-10% DMSO at 1×10⁷ cells/ml. Aliquots of cells were frozen at −80C in a Nalgene Mr. Frosty container (Nalgene Labware, Rochester, NY) before final storage in liquid nitrogen.

PBMC Stimulation: Cryopreserved PBMC aliquots were rapidly thawed in a 37° C. water bath, mixed with an equal volume of RPMI-1640 media warmed to 37° C., and then centrifuged at 400 g for 5 minutes. Cell pellets were washed with 2 mL of RPMI-1640, resuspended in 150 μL RPMI-1640 and analyzed by Trypan Blue exclusion to evaluate cell viability, and then supplemented with RPMI-1640 to a final working concentration of ~3×10⁶ viable cells/mL. Samples that had cell viabilities ≤70% were excluded from analysis. PBMCs were plated in 6 well cell culture plates at a concentration of 1×10⁶ to 2×10⁶ viable cells/well as specified by different assay types, and then stimulated with 10 ng/mL phorbol 12-myristate13-acetate (PMA, Sigma P1585) and 1 μg/mL ionomycin (STEM CELL 73722) or 1 μg/mL of the indicated peptide or peptide pools (BEI NR-52402) at 37° C. for the specified times.

Flow cytometry: PBMCs aliquots suspended in AIM V cell culture media (2×10⁶/mL) were cultured overnight in 24 well culture plates overnight before and then stimulated for 24 h with PMA and iomyocin (10 ng/mL and 1 μg/mL, respectively) or a SARS-CoV-2 or HIV-p24 peptide pool (1 μg/mL), with 1 ng/mL IFN-γ transport blocker added 2 h after the start of induction. Following stimulation, PBMCs were pelleted by centrifugation at 500 g for 5 min, PBS washed, and then resuspended in 100 μL of IC Fixation Buffer and Permeabilization Buffer (eBioscience 00-8222-49 and 00-8333) for 10 min, then incubated in a PBS/10% BSA solution supplemented with 1 μg/ml of an AlexaFluor488-labeled IFN-γ-specific antibody (eBioscience 50-168-09) for 20 min. Flow cytometry analyses were performed using an Attune Flow Cytometer (Thermo Scientific) gating cells, capturing IFN-γ-positive cell signal in the FITC/GFP channel, and analyzing and quantifying captured data with FlowJo software (v10.04).

IGRA ELISAs: PBMCs (2×10⁴) were cultured for the indicated times at 37° C. in X mL RPMI-1640 media supplemented with a 1 μg/mL SARS-COV-2 peptide pool (BEI NR-52402), PMA and ionomycin (10 ng/ml and 1 μg/ml), or no added material, with an RPMI only well included as a negative control. Culture supernatants were pipetted from each well and stored at −80° C. for future ELISA analysis.

After incubation, the media was pipetted from wells into a new 98 well plate. 1 g/mL final concentration of SARS-COV-2 peptide pool was added to the stimulation group at this time. At 4, 6, 8, 10, 12 and 24 hours, the supernatant was removed and stored at −80° C. for future ELISA.

IGRA ELISA plates were generated by incubating 96 well MaxiSorp plates (Nunc 44-2404-21) with 100 μL of 1 μg/ml PBS solution of human IFN-γ-specific antibody (Endogen, M700-A) overnight at 4° C. These plates were then washed 6 times with PBS/0.05% Tween 20 (PBST), blocked with 200 μl of 1% BSA/PBS for 1 h at room temperature, and then PBST washed, dried, and stored at 4° C. until use. Cryopreseved PBMC culture supernatant aliquots were thawed and transferred to assay plates in triplicate (50 μL/well) and incubated at room temperature for 1 h. 50 μl of IFN-γ-biotin-labeled antibody (Endogen, M-701B) diluted at 1:1000 in 2% FBS/1×PBS was added to each well and incubated at room temperature for 1 hour. Plates were washed and dried before pipetting 50 μl/well of Poly-HRP streptavidin (Pierce, N200) diluted at 1:5000 in 1% BSA/1×PBS and incubated at room temperature for 30 minutes in the dark. Afterward, the plate was washed and dried for a final time. 100 μl/well of 3,3′,5,5′-Tetramethylbenzidine (TMB, Thermo Scientific 34029) solution was added, and color development was observed. After adequate color development (~10 minutes) 50 μl/well of stop solution (2.5 N H₂SO₄) was added and plates were read at OD450.

ELISPOT: Filter Screen Plates (Millipore MAIPS4510) were coated with anti-human IFN-γ (Endogen, M700-A, 1 mg/ml) at 1 μg/ml and stored overnight at 4° C. The following day the plate was washed 6 times with washing buffer (1×PBS+1:2000 diluted Tween 20) and tapped dry. Wells were blocked with 200 μl of 1% BSA/1×PBS for 1 hour at room temperature. 2×10⁵ PBMC were then seeded into plates and stimulated with PMA-ionomycin (10 ng/ml and 1 μg/ml), SARS-CoV-2 Spike peptide pool (1 μg/mL) or HIV-p24 peptide (1 μg/mL). 100 μl of IFN-7-biotin-labeled antibody (Endogen, M-701B) diluted at 1:1000 in 1% BSA/1×PBS was added to each well and incubated at room temperature for 1 hour. Plates were washed and dried before pipetting 100 μl/well of Poly-HRP streptavidin (Pierce, N200) diluted at 1:5000 in 1% BSA/1×PBS and incubated at room temperature for 30 minutes in the dark. Then 100 μL/well of 3-Amino-9-ethylcarbazole (AEC, BD 557630) were added and incubated at room temperature for 15 min. The whole plate was washed with DI water and the bottom was separated to be dry completely overnight. The spots were the scanned by CTL-Immunospot S6 Universal Analyzer (ImmunoSpot) and counted by Double-Color ELISPOT Enzymatic software (ImmunoSpot).

### Chip Fabrication

The silicon wafer with the microfluidic design was fabricated based on previously described methods. Polydimethylsiloxane (PDMS) molds of the design were fabricated from the silicon wafer (FIG. 4). The PDMS elastomer was mixed with a curing agent at a 10:1 ratio and pour over the silicon wafer. The curing agent allows for the elastomer to crosslink and form a rigid structure that will solidify into a complete chip. To accelerate the rate at which the PDMS solidifies, the PDMS mold is placed in an oven at 60° C. for 5 hours. After the elastomer has completely solidified, the molds are removed from the silicon wafer to be used for chip assembly. Plasma treatment of a PDMS chip and a 1 mm glass slide allows for the formation of silanol functional groups that can form strong covalent bonds with each other to create a fluid-tight seal that forms that microfluidic channel.

### On-Chip ELISpot Assays:

PMA-ionomycin (10 ng/ml and 1 μg/ml), SARS-CoV-2 Spike peptide pool (1 μg/mL) or HIV-p24 peptide (1 μg/mL) were added into 25 μL whole blood then incubated at 37° C. for 4 hours. The blood samples were fixed with IC Fixation Buffer (eBioscience™ 00-8222-49) and Permeabilization Buffer (eBioscience™ 00-8333) at 25° C. for 20 min then stained with 1 μg/ml anti-IFN-7-Alexa488 (eBioscience 50-168-09) and 0.1 μg/mL Hoechst 33342 at 25° C. for 20 min. On-chip detection were performed as described above.

Image capture and analysis: Images of PBMCs attached microfluidic chamber were obtained using an EVOS™ M5000 Imaging System, Invitrogen by Thermo Fisher Scientific, Madrid, Spain, Scale bar: 300 μm. Images (10×) of the stained PBMCs are representative of the total cell population and the IFN-γ positive cells. The green fluorescence signal was obtained, when Alexa 488 binds to intracellular IFN-γ. The blue fluorescence signal from Hoechst 33342 represents the total cells counts. All the experiments were conducted in triplicate. Each time four different random areas from the microfluidic chamber were chosen to obtain the images. All data acquired on EVOS™ M5000 Imaging System were analyzed using the ImageJ software.

Cell counting: The total cell counts and IFN-γ positive cells ratio were quantified using the National Institutes of Health (NIH) Image J image-analysis software. The images were converted to 8-bit greyscale. The lower threshold value was set to 70 and the higher threshold value was set to 255. The cell counts were analyzed with the size range from 1 to 100 (Pixel) and circularity 0.00-1.00.

The following references are incorporated by reference in their entirety for all purposes.
1 Eyre, D. W. et al. Effect of Covid-19 Vaccination on Transmission of Alpha and Delta Variants. New England Journal of Medicine 386, 744-756, doi:10.1056/NEJMoa2116597 (2022).
2 Singer, S. R. et al. Effectiveness of BNT162b2 mRNA COVID-19 vaccine against SARS-CoV-2 variant Beta (B.1.351) among persons identified through contact tracing in Israel: A prospective cohort study. eClinicalMedicine 42, doi:10.1016/j.eclinm.2021.101190 (2021).
3 Ranzani, O. T. et al. Effectiveness of the CoronaVac vaccine in older adults during a gamma variant associated epidemic of covid-19 in Brazil: test negative case-control study. BMJ 374, n2015, doi:10.1136/bmj.n2015 (2021).
4 Andrews, N. et al. Covid-19 Vaccine Effectiveness against the Omicron (B.1.1.529) Variant. New England Journal of Medicine, doi:10.1056/NEJMoa2119451 (2022).
5 Starr, T. N. et al. Prospective mapping of viral mutations that escape antibodies used to treat COVID-19. Science 371, 850-854 (2021).
6 Wilhelm, A. et al. Reduced Neutralization of SARS-CoV-2 Omicron Variant by Vaccine Sera and Monoclonal Antibodies. medRxiv, doi:10.1101/2021.12.07.21267432 (2021).
7 Goldberg, Y. et al. Protection and waning of natural and hybrid COVID-19 immunity. medRxiv, doi:10.1101/2021.12.04.21267114 (2021).
8 Hoffmann, M. et al. The Omicron variant is highly resistant against antibody-mediated neutralization—implications for control of the COVID-19 pandemic. Cell, doi:10.1016/j.cell.2021.12.032 (2021).
9 Liu, L. et al. Striking antibody evasion manifested by the Omicron variant of SARS-CoV-2. bioRxiv (2021).
10 Tang, Y.-W., Schmitz, J. E., Persing, D. H. & Stratton, C. W. Laboratory diagnosis of COVID-19: current issues and challenges. Journal of clinical microbiology 58, e00512-00520 (2020).
11 D'Cruz, R. J., Currier, A. W. & Sampson, V. B. Laboratory Testing Methods for Novel Severe Acute Respiratory Syndrome-Coronavirus-2 (SARS-CoV-2). Front Cell Dev Biol 8, 468, doi:10.3389/fcell.2020.00468 (2020).
12 Petersen, L. R. et al. Lack of antibodies to severe acute respiratory syndrome coronavirus 2 (SARS-CoV-2) in a large cohort of previously infected persons. Clinical Infectious Diseases 73, e3066-e3073 (2021).
13 Demey, B. et al. Dynamic profile for the detection of anti-SARS-CoV-2 antibodies using four immunochromatographic assays. J Infect 81, e6-e10, doi:10.1016/j.jinf.2020.04.033 (2020).
14 Hodgson, S. H. et al. What defines an efficacious COVID-19 vaccine? A review of the challenges assessing the clinical efficacy of vaccines against SARS-CoV-2. The lancet infectious diseases 21, e26-e35 (2021).
15 Jung, J. H. et al. SARS-CoV-2-specific T cell memory is sustained in COVID-19 convalescent patients for 10 months with successful development of stem cell-like memory T cells. Nat Commun 12, 4043, doi:10.1038/s41467-021-24377-1 (2021).
16 Le Bert, N. et al. SARS-CoV-2-specific T cell immunity in cases of COVID-19 and SARS, and uninfected controls. Nature 584, 457-462, doi:10.1038/s41586-020-2550-z (2020).
17 Ogbe, A. et al. T cell assays differentiate clinical and subclinical SARS-CoV-2 infections from cross-reactive antiviral responses. Nat Commun 12, 2055, doi:10.1038/s41467-021-21856-3 (2021).
18 Ng, O. W. et al. Memory T cell responses targeting the SARS coronavirus persist up to 11 years post-infection. Vaccine 34, 2008-2014, doi:10.1016/j.vaccine.2016.02.063 (2016).
19 Zhao, J. et al. Recovery from the Middle East respiratory syndrome is associated with antibody and T-cell responses. Sci Immunol 2, doi:10.1126/sciimmunol.aan5393 (2017).
20 Gallais, F. et al. Intrafamilial Exposure to SARS-CoV-2 Associated with Cellular Immune Response without Seroconversion, France. Emerg Infect Dis 27, doi:10.3201/eid2701.203611 (2021).
21 Sattler, A. et al. Impaired humoral and cellular immunity after SARS-CoV2 BNT162b2 (Tozinameran) prime-boost vaccination in kidney transplant recipients. The Journal of Clinical Investigation (2021).
22 Herishanu, Y. et al. Efficacy of the BNT162b2 mRNA COVID-19 vaccine in patients with chronic lymphocytic leukemia. Blood, The Journal of the American Society of Hematology 137, 3165-3173 (2021).
23 Furer, V. et al. (BMJ Publishing Group Ltd, 2021).
24 Achiron, A. et al. Humoral immune response to COVID-19 mRNA vaccine in patients with multiple sclerosis treated with high-efficacy disease-modifying therapies. Therapeutic Advances in Neurological Disorders 14, 17562864211012835 (2021).
25 Madelon, N. et al. Robust T cell responses in anti-CD20 treated patients following COVID-19 vaccination: a prospective cohort study. Clinical Infectious Diseases (2021).
26 Stamatatos, L. et al. mRNA vaccination boosts cross-variant neutralizing antibodies elicited by SARS-CoV-2 infection. Science (2021).
27 Poudel, S. et al. Highly mutated SARS-CoV-2 Omicron variant sparks significant concern among global experts—What is known so far? Travel Med Infect Dis 45, 102234, doi:10.1016/j.tmaid.2021.102234 (2021).
28 Cele, S. et al. SARS-CoV-2 Omicron has extensive but incomplete escape of Pfizer BNT162b2 elicited neutralization and requires ACE2 for infection. medRxiv, doi:10.1101/2021.12.08.21267417 (2021).
29 Keeton, R. et al. SARS-CoV-2 spike T cell responses induced upon vaccination or infection remain robust against Omicron. medRxiv, doi:10.1101/2021.12.26.21268380 (2021).
30 Tormo, N. et al. Commercial Interferon-gamma release assay to assess the immune response to first and second doses of mRNA vaccine in previously COVID-19 infected versus uninfected individuals. Diagnostic Microbiology and Infectious Disease, doi:10.1016/j.diagmicrobio.2021.115573 (2021).
31 Van Praet, J. T. et al. Dynamics of the cellular and humoral immune response after BNT162b2 mRNA Covid-19 vaccination in Covid-19 naive nursing home residents. J Infect Dis, doi:10.1093/infdis/jiab458 (2021).
32 Kruttgen, A. et al. Evaluation of the QuantiFERON SARS-CoV-2 interferon-release assay in mRNA-1273 vaccinated health care workers. J Virol Methods 298, 114295, doi:10.1016/j.jviromet.2021.114295 (2021).
33 Jaganathan, S. et al. Preliminary Evaluation of QuantiFERON SARS-CoV-2 and QIAreach Anti-SARS-CoV-2 Total Test in Recently Vaccinated Individuals. Infect Dis Ther 10, 2765-2776, doi:10.1007/s40121-021-00521-8 (2021).
34 Kruse, M. et al. Performance of the T-SPOTO.COVID test for detecting SARS-CoV-2-responsive T cells. Int J Infect Dis 113, 155-161, doi:10.1016/j.ijid.2021.09.073 (2021).
35 Demaret, J. et al. Severe SARS-CoV-2 patients develop a higher specific T-cell response. Clin Transl Immunology 9, e1217, doi:10.1002/cti2.1217 (2020).
36 Schwarzkopf, S. et al. Cellular Immunity in COVID-19 Convalescents with PCR-Confirmed Infection but with Undetectable SARS-CoV-2-Specific IgG. Emerging Infectious Diseases 27, 122-129, doi:10.3201/eid2701.203772 (2021).
37 Cassaniti, I. et al. SARS-CoV-2 specific T-cell immunity in COVID-19 convalescent patients and unexposed controls measured by ex vivo ELISpot assay. Clin Microbiol Infect 27, 1029-1034, doi:10.1016/j.cmi.2021.03.010 (2021).
38 Murugesan, K. et al. Interferon-gamma Release Assay for Accurate Detection of Severe Acute Respiratory Syndrome Coronavirus 2 T-Cell Response. Clin Infect Dis 73, e3130-e3132, doi:10.1093/cid/ciaa1537 (2021).
39 Petrone, L. et al. A whole blood test to measure SARS-CoV-2-specific response in COVID-19 patients. Clin Microbiol Infect 27, 286 e287-286 e213, doi:10.1016/j.cmi.2020.09.051 (2021).
40 Echeverria, G. et al. Pre-existing T-cell immunity to SARS-CoV-2 in unexposed healthy controls in Ecuador, as detected with a COVID-19 Interferon-Gamma Release Assay. Int J Infect Dis 105, 21-25, doi:10.1016/j.ijid.2021.02.034 (2021).
41 Stambaugh, A. et al. Optofluidic multiplex detection of single SARS-CoV-2 and influenza A antigens using a novel bright fluorescent probe assay. Proc Natl Acad Sci USA 118, doi:10.1073/pnas.2103480118 (2021).
42 Gao, Z. et al. Machine-Learning-Assisted Microfluidic Nanoplasmonic Digital Immunoassay for Cytokine Storm Profiling in COVID-19 Patients. ACS Nano, doi:10.1021/acsnano.1c06623 (2021).
43 Song, Q. et al. Point-of-care testing detection methods for COVID-19. Lab Chip 21, 1634-1660, doi:10.1039/d01c01156h (2021).
44 Heggestad, J. T. et al. Multiplexed, quantitative serological profiling of COVID-19 from blood by a point-of-care test. Science advances 7, eabg4901 (2021).
45 Chen, Q., Li, Y., Liu, Y., Xu, W. & Zhu, X. Exosomal Non-coding RNAs-Mediated Crosstalk in the Tumor Microenvironment. Front Cell Dev Biol 9, 646864, doi:10.3389/fcell.2021.646864 (2021).
46 Tan, S. et al. Exosomal miRNAs in tumor microenvironment. Journal of Experimental & Clinical Cancer Research 39 (2020).
47 Zhang, L. et al. Microenvironment-induced PTEN loss by exosomal microRNA primes brain metastasis outgrowth. Nature 527, 100-104, doi:10.1038/nature15376 (2015).
48 Neviani, P. et al. Natural Killer-Derived Exosomal miR-186 Inhibits Neuroblastoma Growth and Immune Escape Mechanisms. Cancer Res 79, 1151-1164, doi:10.1158/0008-5472.CAN-18-0779 (2019).
49 Seo, N. et al. Activated CD8(+) T cell extracellular vesicles prevent tumour progression by targeting of lesional mesenchymal cells. Nat Commun 9, 435, doi:10.1038/s41467-018-02865-1 (2018).
50 Yavin, E. & Yavin, Z. Attachment and culture of dissociated cells from rat embryo cerebral hemispheres on polylysine-coated surface. The Journal of cell biology 62, 540 (1974).
51 Tsang, M., Gantchev, J., Ghazawi, F. M. & Litvinov, I. V. Protocol for adhesion and immunostaining of lymphocytes and other non-adherent cells in culture. Biotechniques 63, 230-233 (2017).
52 Shipkova, M. & Wieland, E. Surface markers of lymphocyte activation and markers of cell proliferation. Clin Chim Acta 413, 1338-1349, doi:10.1016/j.cca.2011.11.006 (2012).
53 Redmond, W. L., Ruby, C. E. & Weinberg, A. D. The role of OX40-mediated co-stimulation in T-cell activation and survival. Crit Rev Immunol 29, 187-201, doi:10.1615/critrevimmunol.v29.i3.10 (2009).
54 Dawicki, W. & Watts, T. H. Expression and function of 4-1BB during CD4 versus CD8 T cell responses in vivo. European Journal of Immunology 34, 743-751, doi:https://doi.org/10.1002/eji.200324278 (2004).
55 Tan, J. T., Whitmire, J. K., Ahmed, R., Pearson, T. C. & Larsen, C. P. 4-1BB Ligand, a Member of the TNF Family, Is Important for the Generation of Antiviral CD8 T Cell Responses. The Journal of Immunology 163, 4859 (1999).
56 Hagen, J. et al. Comparative Multi-Donor Study of IFNγ Secretion and Expression by Human PBMCs Using ELISPOT Side-by-Side with ELISA and Flow Cytometry Assays. Cells 4, 84-95, doi:10.3390/cells4010084 (2015).
57 Mallone, R. et al. Isolation and preservation of peripheral blood mononuclear cells for analysis of islet antigen-reactive T cell responses: position statement of the T-Cell Workshop Committee of the Immunology of Diabetes Society. Clinical and Experimental Immunology 163, 33-49, doi:10.1111/j.1365-2249.2010.04272.x (2010).
58 Nilsson, C. et al. Optimal blood mononuclear cell isolation procedures for gamma interferon enzyme-linked immunospot testing of healthy Swedish and Tanzanian subjects. Clin Vaccine Immunol 15, 585-589, doi:10.1128/cvi.00161-07 (2008).
59 Oguntoye, I. O. et al. Silicon Nanodisk Huygens Metasurfaces for Portable and Low-Cost Refractive Index and Biomarker Sensing. ACS Applied Nano Materials 5, 3983-3991, doi:10.1021/acsanm.1c04443 (2022).
60 Organization WH. Tuberculosis & HIV. Accessed Jun. 4, 2023. https://www.who.int/teams/global-hiv-hepatitis-and-stis-programmes/hiv/treatment/tuberculosis-hiv
61. Clark R A, Mukandavire C, Portnoy A, et al. The impact of alternative delivery strategies for novel tuberculosis vaccines in low-income and middle-income countries: a modelling study. The Lancet Global Health. 2023; 11(4):e546-e555.
62. Castro K G, Goldberg S, Jereb J A, LoBue P, Mazurek G H, Vernon A. Updated guidelines for using interferon gamma release assays to detect Mycobacterium tuberculosis infection—United States, 2010. 2010;
63. Szturmowicz M, Broniarek-Samson B, Demkow U. Prevalence and risk factors for latent tuberculosis in polish healthcare workers: the comparison of tuberculin skin test and interferon-gamma release assay (IGRA) performance. Journal of Occupational Medicine and Toxicology. 2021; 16:1-12.
64. Almeida Santos J, Duarte R, Nunes C. Tuberculin skin test and predictive host factors for false-negative results in patients with pulmonary and extrapulmonary tuberculosis. The Clinical Respiratory Journal. 2020; 14(6):541-548.
65. Bruchfeld J, Correia-Neves M, Kallenius G. Tuberculosis and HIV coinfection. Cold Spring Harbor perspectives in medicine. 2015; 5(7):a017871.
66. Organization WH. Global Tuberculosis Report 2022. Accessed Jun. 9, 2023. https://www.who.int/teams/global-tuberculosis-programme/tb-reports/global-tuberculosis-report-2022
67. Organization WH. Global tuberculosis report 2021: supplementary material. 2022;
68. Green A M, DiFazio R, Flynn J L. IFN-γ from CD4 T cells is essential for host survival and enhances CD8 T cell function during Mycobacterium tuberculosis infection. The Journal of Immunology. 2013; 190(1):270-277.
69. Tungsattayathitthan U, Boonsopon S, Tesavibul N, Dharakul T, Choopong P. Interferon-gamma release assays in tuberculous uveitis: a comprehensive review. International Journal of Ophthalmology. 2022; 15(9):1520.
70. Poloni C, Schonhofer C, Ivison S, Levings M K, Steiner T S, Cook L. T-cell activation-induced marker assays in health and disease. Immunology and Cell Biology. 2023;
71. Gramaglia I, Weinberg A D, Lemon M, Croft M. Ox-40 ligand: a potent costimulatory molecule for sustaining primary CD4 T cell responses. The Journal of Immunology. 1998; 161(12):6510-6517.
72. Wang C, Lin G H, McPherson A J, Watts T H. Immune regulation by 4-1BB and 4-1BBL: complexities and challenges. Immunological reviews. 2009; 229(1):192-215.
73. Hermann-Kleiter N, Baier G. NFAT pulls the strings during CD4+ T helper cell effector functions. Blood, The Journal of the American Society of Hematology. 2010; 115(15):2989-2997.
74. Aarts M, Abboud S, Abdulkarim F, et al. Abramson, J., I. Smirnova, V. Kasho, G. Verner, S. Iwata and HR Kaback, The lactose permease of Escherichia coli: overall structure, the sugar-binding site and the alternating access model for transport (555) 96 Abu-bakr, A. see M. Saidijam (555) 170. transport. 555:96.
75. Herndler-Brandstetter D, Schwaiger S, Veel E, et al. CD25-expressing CD8+ T cells are potent memory cells in old age. The Journal of Immunology. 2005; 175(3):1566-1574.
76. Bremser A, Brack M, Izcue A. Higher sensitivity of Foxp3+ Treg compared to Foxp3-conventional T cells to TCR-independent signals for CD69 induction. PLoS One. 2015; 10(9):e0137393.
77. Yellin M J, Sippel K, Inghirami G, et al. CD40 molecules induce down-modulation and endocytosis of T cell surface T cell-B cell activating molecule/CD40-L. Potential role in regulating helper effector function. Journal of immunology (Baltimore, Md: 1950). 1994; 152(2):598-608.
78. Komiya K, Ariga H, Nagai H, et al. Impact of peripheral lymphocyte count on the sensitivity of 2 IFN-γ release assays, QFT-G and ELISPOT, in patients with pulmonary tuberculosis. Internal Medicine. 2010; 49(17):1849-1855.
79. Jones B E, Oo M M, Taikwel E K, et al. CD4 cell counts in human immunodeficiency virus—negative patients with tuberculosis. Clinical Infectious Diseases. 1997; 24(5):988-991.
80. Kony S, Hane A, Larouze B, et al. Tuberculosis-associated severe CD4+ T-lymphocytopenia in HIV-seronegative patients from Dakar. Journal of Infection. 2000; 41(2):167-171.
81. Nguyen D T, Teeter L D, Graves J, Graviss E A. Characteristics associated with negative interferon-7 release assay results in culture-confirmed tuberculosis patients, Texas, USA, 2013-2015. Emerging infectious diseases. 2018; 24(3):534.
82. Pan L, Jia H, Liu F, et al. Risk factors for false-negative T-SPOT. TB assay results in patients with pulmonary and extra-pulmonary TB. Journal of Infection. 2015; 70(4):367-380.
83. Hougardy J-M, Place S, Hildebrand M, et al. Regulatory T cells depress immune responses to protective antigens in active tuberculosis. American journal of respiratory and critical care medicine. 2007; 176(4):409-416.
84. Chen X, Zhou B, Li M, et al. CD4+ CD25+ FoxP3+ regulatory T cells suppress Mycobacterium tuberculosis immunity in patients with active disease. Clinical immunology. 2007; 123(1):50-59.
85. Mills K H. Regulatory T cells: friend or foe in immunity to infection? Nature Reviews Immunology. 2004; 4(11):841-855.
86. Barham M S, Whatney W E, Khayumbi J, et al. Activation-Induced Marker Expression Identifies Mycobacterium tuberculosis-Specific CD4 T Cells in a Cytokine-Independent Manner in HIV-Infected Individuals with Latent Tuberculosis. Immunohorizons. 2020; 4(10):573-584.

What is claimed is:

1. A method of identifying pathogen-specific T-cell activation, comprising the steps of:
a) obtaining a biological sample from a subject;
b) introducing an incubation mixture into the biological sample, wherein the incubation mixture comprises (i) peptides of a pathogen of interest or peptides of a vaccine of interest, and (ii) an antibody binding specific to activated T-cells upon stimulation by the peptides of the pathogen of interest or by the peptides of the vaccine of interest;
c) detecting presence of the activated T-cell in the biological sample from step b);
wherein the antibody in step b) is conjugated with an enzyme or a fluorescent molecule.

2. The method of claim 1, wherein the biological sample is a whole blood sample.

3. The method of claim 2, wherein the biological sample does not undergo isolation of peripheral blood mononuclear cells (PBMCs) or removal of red blood cells.

4. The method of claim 2, wherein the amount of the whole blood sample is less than 1 mL.

5. The method of claim 2, further comprising, after step b), the following step b-1):
b-1) obtaining T-cells in the whole blood sample by CD4 and CD8 specific antibodies.

6. The method of claim 1, wherein the pathogen of interest is SARS-CoV-2, HIV, M. tuberculosis, Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, or Trypanosoma cruzi.

7. The method of claim 6, wherein the peptides of the pathogen of interest include peptides from SARS-CoV-2 spike peptide pool, M. tuberculosis, BEI NR-52402, Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, Trypanosoma cruzi, or tumor specific antigen peptide from NY-ESO-1, HER2, PSA, TRP-2, EpCAM, GPC3, mesothelin (MSLN), MUC1, EGFR, OX-40, CD59, LAG-3, TIM3, and IL-12R, CD28, CD57, KIR, KLRG-1, CD27, PD-1, CTLA-4.

8. The method of claim 1, wherein in step b) further comprising introducing at least one of the following to the biological sample: phorbol 12-myristate13-acetate (PMA) and ionomycin.

9. The method of claim 1, wherein the antibody is specific against OX-40 or 4-1BB.

10. The method of claim 1, wherein the antibody binds against IFN-7 and is M700-A from Endogen.

11. (canceled)

12. A point-of-care kit for identifying pathogen-specific T-cell response, comprising:
a) a microfluidic device having a plurality of microfluidic channels connecting a sample inlet to a detection chamber;
b) wherein the detection chamber is coated with poly-lysine.

13. The point-of-care kit of claim 12, further comprising an incubation container having peptides from a pathogen of interest or peptides from a vaccine of interest, wherein a biological sample is introduced into the incubation container.

14. The point-of-care kit of claim 13, wherein the detection chamber further comprising at least one of: anti-human IFN-7 antibodies, anti-4-1BB antibodies, and anti-OX-40 antibodies, wherein the antibodies are conjugated with an enzyme or a fluorescent molecule.

15. The point-of-care device of claim 13, wherein the pathogen of interest is SARS-CoV-2, HIV, M. tuberculosis, Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, and Trypanosoma cruzi.

16. The point-of-care device of claim 13, wherein the vaccine of interest is a vaccine against SARS-CoV-2, M. tuberculosis, Cytomegalovirus (CMV), Influenza, Respiratory syncytial virus (RSV), herpes simplex virus (HSV), Hepatitis B virus (HBV), Epstein-Barr virus (EBV), Listeria, Salmonella, Plasmodium, Toxoplasma gondii, or Trypanosoma cruzi.

17. The point-of-care kit of claim 13, wherein the incubation container further comprising phorbol 12-myristate13-acetate (PMA) and ionomycin.

18. A method of identifying pathogen-specific T-cell activation, comprising the steps of:
a) obtaining a biological sample from a subject;
b) introducing an incubation mixture into the biological sample, wherein the incubation mixture comprises (i) peptides of a pathogen of interest or peptides of a vaccine of interest, and (ii) an antibody specific to a cytokine or a surface marker, wherein the cytokine is secreted by T-cells in the biological sample upon stimulation by the peptides of the pathogen of interest or by the peptides of the vaccine of interest; and
c) detecting presence of the cytokine or the surface marker at a reaction chamber of the point-of-care kit of claim 12;
wherein the cytokine- or surface marker-specific antibody is conjugated with an enzyme or a fluorescent molecule.

19. The method of claim 18, wherein the cytokine is IL-2, IL-4, IL-17 or TNFα.

20. The method of claim 18, wherein the surface marker is at least a portion of OX-40, 4-1BB, CD59, LAG-3, TIM3, and IL-12R, CD28, CD57, KIR, KLRG-1, CD27, PD-1, CTLA-4, IFN-7, IL-2, IL-10, or TNF-α.

21. The method of claim 18, wherein the mixture in step b) is introduced into the reaction chamber at a flow rate between 5 μl/min to 20 μl/min.
